(19) United States
(12) Patent Application Publication
(10) Pub. No.: US 2014/0039506 A1
(43) Pub. Date: Feb. 6, 2014

(54) SUBTALAR JOINT PROSTHESIS AND INSTALLATION DEVICE

(71) Applicant: Innovative Biological Implant Solutions LLC, Moore, OK (US)

(72) Inventors: Vytautas Ringus, Norman, OK (US); Matthew L. Landsberger, Moore, OK (US)

(73) Assignee: Innovative Biological Implant Solutions LLC, Moore, OK (US)

(21) Appl. No.: 13/956,901

(22) Filed: Aug. 1, 2013

Related U.S. Application Data
(60) Provisional application No. 61/678,474, filed on Aug. 1, 2012.

(51) Int. Cl. A61F 2/42; A61B 17/16; A61B 17/66

(52) U.S. Cl. CPC A61F 2/4202; A61B 17/66; A61B 17/1682
USPC 606/82; 623/21.18; 606/90

(57) ABSTRACT

A first implant may be configured for attachment to a talus bone. A second implant may be configured for attachment to a calcaneus bone. The first implant attached to the talus bone can move in relation to the second implant attached to the calcaneus bone. A sheet can be disposed between the first implant and the second implant to facilitate relative motion between the first implant and the second implant. The sheet may provide a curvilinear surface that engages a curvilinear surface of the first implant or a curvilinear surface of the second implant to facilitate the relative motion. A set of tools may be used to distract the talus bone from the calcaneus, which may be used for insertion of the implants.

### RELATED APPLICATIONS

This application claims priority to U.S. Provisional Patent Application No. 61/678,474 filed on Aug. 1, 2012, entitled “ARTIFICIAL PROSTHESIS FOR SUBTALAR JOINT,” which is totally incorporated herein by reference.

### BRIEF DESCRIPTION OF THE DRAWINGS

FIG. 1A is a top perspective view of an assembled subtalar joint prosthesis according to the present disclosure.

FIG. 1B is a side view of FIG. 1A.

FIG. 2 is an exploded view of certain aspects of the subtalar joint prosthesis of FIG. 1A.

FIGS. 3A is a top plan view of an assembled subtalar joint prosthesis according to the present disclosure.

FIG. 3B is a cross-sectional view taken along line of 3B-3B of FIG. 3A.

FIG. 3C is a cross-sectional view taken along line of 3C-3C of FIG. 3B.

FIG. 4A is a top perspective view of a talar implant according to the present disclosure.

FIG. 4B is a bottom plan view of FIG. 4A.

FIG. 5A is a top plan view of a sheet according to the present disclosure.

FIG. 5B is a bottom perspective view of FIG. 5A.

FIG. 6A is a top perspective view of a calcaneal implant according to the present disclosure.

FIG. 6B is a bottom perspective view of FIG. 6A.

FIG. 7A is a top perspective view of a sheet interlocking with a calcaneal implant according to the present disclosure.

FIG. 7B is a bottom perspective view of the sheet of FIG. 7A.

FIG. 7C is a top perspective view of the calcaneal implant of FIG. 7A.

FIG. 8A is a first perspective view of a distracter according to the present disclosure.

FIG. 8B is a second perspective view of a distracter of FIG. 8A.

FIG. 9 is an exploded view of the distracter of FIGS. 8A-B.

FIG. 10A is a front view in the undistracted position of the distracter according to the present disclosure.

FIG. 10B is a front view in the distracted position of the distracter of FIG. 10A.

FIG. 11A is a front view of a wrench in the neutral state of FIG. 9.

FIG. 11B is a front view of a wrench in the activated state of FIG. 9.

FIG. 12 is a perspective view of a front body of FIG. 9.

FIG. 13A is a front perspective view of a main body of FIG. 9.

FIG. 13B is a back perspective view of a main body of FIG. 9.

FIG. 13C is a close-up view of a main body of FIG. 9.

FIG. 14 is a perspective view of a cover plate of FIG. 9.

FIG. 15 is a perspective view of a main gear of FIG. 9.

FIG. 16 is a perspective view of a wrench gear of FIG. 9.

FIG. 17A is a first perspective view of a locking wrench gear of FIG. 9.

FIG. 17B is a second perspective view of a locking wrench gear of FIG. 9.

FIG. 18 is a perspective view of a pin guide of FIG. 9.

FIG. 19A is a first perspective view of a bias element pin of FIG. 9.

FIG. 19B is a second perspective view of a bias element pin of FIG. 9.

FIG. 20 is a side view of a bias element of FIG. 9.

FIG. 21 is a perspective view of a button of FIG. 9.

FIG. 22 is a perspective view of a handle of FIG. 9.

FIG. 23A is a first perspective view of a talus bone cutter guide according to the present disclosure.

FIG. 23B a second perspective view of a talus bone cutter guide of FIG. 23A.

FIG. 24A is a perspective view of the talus bone cutter of FIG. 23A attached to a talus bone.

FIG. 24B is a perspective view of the talus bone cut with the talus bone cutter guide of FIG. 23A.

FIG. 25A is a first perspective view of a dummy plate according to the present disclosure.

FIG. 25B is a second perspective view of a dummy plate of FIG. 25A.

FIG. 26 is a perspective view of the dummy plate of FIG. 25A attached to the talus bone.

FIG. 27 is a perspective view of a first calcaneus cutter guide according to the present disclosure.

FIG. 28A is a perspective view of the first calcaneus bone cutter of FIG. 27 attached to the calcaneus.

FIG. 28B is a perspective view of the calcaneus cut with the calcaneus cutter guide of FIG. 27.

FIG. 29A is a first perspective view of a coronal plane guide rail of FIG. 27.

FIG. 29B is a second perspective view of a coronal plane guide rail of FIG. 29A.

FIG. 30 is a perspective view of a sagittal plane guide rail of FIG. 27.

FIG. 31 is a perspective view of a calcaneus cutter guide bottom of FIG. 27.

FIG. 32 is a perspective view of a calcaneus cutter guide top of FIG. 27.

FIG. 33 is a perspective view of a second calcaneus cutter guide according to the present disclosure.

FIG. 34A is a perspective view of the second calcaneus cutter guide of FIG. 33 attached to the calcaneus.

FIG. 34B is a perspective view of the calcaneus cut with the second calcaneus cutter guide of FIG. 33 and the first calcaneus cutter guide of FIG. 27.

FIG. 35A is a perspective view of an angled guide portion of FIG. 33.

FIG. 35B is a perspective view (almost side view) of the angled guide portion of FIG. 35A.

FIG. 36A is a first perspective view of a base plate of FIG. 33.

FIG. 36B is a second perspective view of the base plate of FIG. 36A.

FIG. 37A is a front view of the second calcaneus cutter guide of FIG. 33.

FIG. 37B is a cross-sectional view along lines 37A-37A.

FIG. 38 is a perspective view of a calcaneal implant attached to the calcaneus according to the present disclosure.

FIG. 39 is a bottom view of a talar implant attached to the talus bone according to the present disclosure.

FIG. 40 is a side view looking from the lateral aspect showing the subtalar joint prosthesis installed according to the present disclosure.

FIG. 41 is a side view looking from the lateral aspect showing the subtalar joint distracted by a distracter according to the present disclosure.

FIG. 42 is a perspective view of anatomic planes as used in the present disclosure.

FIG. 43 shows a lateral view of the talus and calcaneus anatomy in a human body as used in the present disclosure.

### DETAILED DESCRIPTION

The present disclosure relates to an artificial joint replacement for the subtalar joint of the human body, which is also known herein as a subtalar joint prosthesis, and a device to distract the talus bone from the calcaneus, which may be used for insertion of the artificial joint replacement.

Over time the subtalar joint can suffer insults from a variety of factors, such as, but not limited to, arthritis, trauma, and infection causing a loss of cartilage, end stage arthrosis, loss of motion, pain, etc. The current standard of care to treat these conditions consists of fixing the talus bone (also known herein as the talus, and also known as astragalus or “ankle bone”) relative to the calcaneus bone (also known herein as the calcaneus, and also known as “heel bone”), such as with a plurality of solid fixed screws, which eliminates substantially all range of motion at the subtalar joint. The disclosed implant device (known also herein as subtalar joint prosthesis) and the disclosed installation device to install a subtalar joint prosthesis may help enable relief of subtalar joint abnormalities while maintaining some range of motion at the subtalar joint. The subtalar joint prosthesis might also be used for reconstructive purposes that may be needed due to traumatic injury or similar and/or different situations.

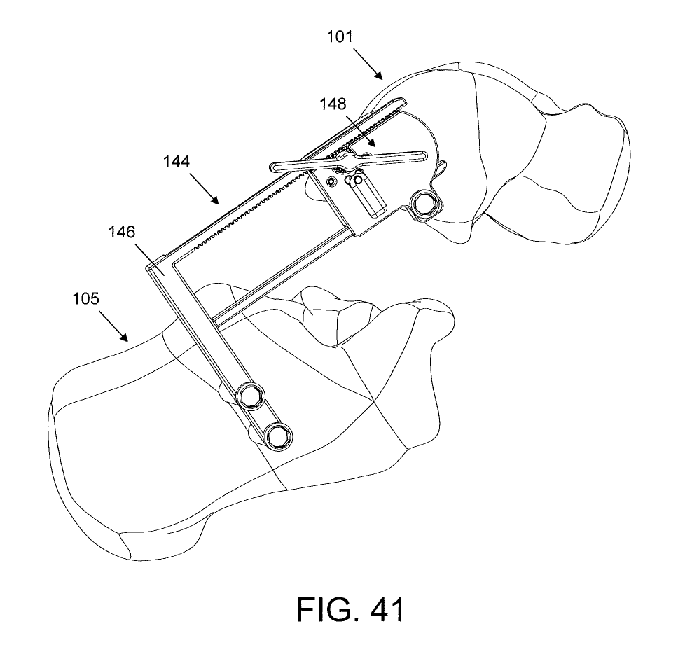

The subtalar joint prosthesis may generally include three main bodies: talar implant (which could also be known as the top plate), calcaneal implant (which could also be known as the bottom plate), and sheet positioned between the talar implant and calcaneal implant. The talar implant can be fixed in place relative to the talus of the foot, the calcaneal implant can be fixed in place relative to the calcaneus of the foot, and the sheet can provide a joint surface by which the talar implant may rotate, translate, and move up and down relative to the calcaneal implant, thus restoring the anatomic motion of the subtalar joint in the transverse, sagittal, and coronal planes. (See FIG. 41.) The talar implant can be contactingly adjacent the talus. The calcaneal implant can be contactingly adjacent the calcaneus. The sheet can be contactingly adjacent both the talus and the calcaneus. Alternatively, the roles of the talar and calcaneal implants may be reversed such that the sheet provides a joint surface by which the calcaneal implant can rotate, translate, and move in relation to the talar implant.

Motion in the subtalar joint prosthesis may include rotation of the talar or calcaneal implant about the axis of the curvilinear surface of the sheet and in the up and down direction relative to the calcaneal or talar implants, respectively. The curvilinear surface facilitates rotation. In addition, the sheet may provide for motion, such as planar translation, substantially parallel to a surface of the calcaneal or talar implant, e.g., when the sheet provides a substantially two-dimensional planar surface that engages a substantially two-dimensional planar surface of the calcaneal or talar implant and the sheet is not fixed in relation to the engaged calcaneal or talar implant. The substantially two-dimensional planar surface facilitates planar translation.

The planar translation may serve to significantly reduce wear rates of the sheet during motion of the subtalar joint prosthesis, thereby increasing the lifespan of the subtalar joint prosthesis as well as reducing potential debris generated by the subtalar joint prosthesis. The planar translation also may provide an additional degree of freedom further enabling kinematic joint motion more similar to that of a native subtalar joint, e.g., a healthy and unreplaced subtalar joint, with normal kinematic motion.

However, due to subtalar joint kinematic complexities and unknown subtalar joint prosthesis wear rates, especially wear rates for the sheet, it may be beneficial to have the sheet fixed in place relative to the calcaneal implant. The fixation of the sheet in relation to the calcaneal implant could be accomplished by adding interlocking features to both the calcaneal implant and sheet, as detailed hereinbelow. Alternatively, the sheet could be fixed with respect to the talar implant when the curvilinear surface of the sheet engages the curvilinear surface of the calcaneal implant.

If the sheet is fixed relative to the calcaneal implant, the planar translation, or motion, of the sheet in relation to the calcaneal implant could be substantially reduced or substantially eliminated. The sheet could also be fixed relative the talar implant to substantially reduce or substantially eliminate planar translation of the sheet in relation to the talar implant.

The sheet may provide a first curvilinear surface that engages the curvilinear surface of the calcaneal implant and a second curvilinear surface that engages the curvilinear surface of the talar implant.

The native subtalar joint may be positioned such that the joint axis can be approximately 45° (or 45 degrees) from the transverse plane when the body is in the upright position with the foot in the transverse plane 90° to the body, and the tibia and fibula are lengthwise in the coronal plane, with the relationship of the foot to the tibia and fibula known as plantigrade neutral. The axis of the subtalar joint prosthesis may be also situated, when implanted in the body, at approximately 45° from the horizontal, or transverse, plane when the talar and calcaneal implants are situated substantially parallel to one another. This orientation of the subtalar joint prosthesis can be done to help the subtalar joint prosthesis enable joint motion similar to that of the native subtalar joint which may help establish a close restoration of anatomic joint kinematics during gait. Of course, the joint axis of the subtalar joint prosthesis may be modified as needed.

Implanting the subtalar joint prosthesis in the body may need a new or unique procedure as well as tools to assist in performance of the procedure. Several bone cuts may be needed to create a bleeding bone surface to which the subtalar joint prosthesis could become affixed as detailed herein, to make room for the subtalar joint prosthesis, as well as to provide surfaces to conform to those surfaces of the subtalar joint prosthesis which may interface with the bones. There may be multiple tools needed to perform the surgical procedure. Some of the tools may be for cutting and shaping the bones and some of the tools may be used to distract the talus bone relative to the calcaneus to make room for the bone cuts as well as subtalar joint prosthesis installation, while preserving other bones, such as the fibula.

A first step in the surgical procedure can be to make a lateral dissection exposing the subtalar joint laterally after clearing soft tissue as necessary. A distracter can be then fastened to the lateral sides of the talus and calcaneus bones, when the lateral approach is used, but other approaches may be used and stay within the scope of the disclosure. The talus and calcaneus bones can then be distracted approximately two inches, or other suitable distance.

A talus bone cutter guide, or talus cutter jig, can then be fastened to the anterolateral undersurface of the talus after removing the anterior osteophytes, also known as the anterior lip. After the talus cutter jig is positioned and set in place, a trial depth gauge can be inserted into a more inferior talus jig slot that defines an approximate depth, approximate rotation, and approximate orientation for a cut in the talus. A substantially flat shaped saw type blade can then be inserted into the talus cutter jig, and the talus bone can be cut substantially flat to conform to the surface of the talar implant which can be substantially parallel with a trial gauge. The dummy plate, mimicking the geometry of the talar implant, can be fastened to the talus. A guide aperture can be used to drill a hole equal to, or slightly larger, than a post of the talar implant. The post of the talar implant may allow for additional long term fixation of the subtalar joint prosthesis as detailed herein.

A first calcaneus cutter guide, or first calcaneus cutter jig, can be then fastened to the lateral side of the calcaneus at the proximal base of the anterior surface. After the first calcaneus cutter jig is positioned and set in place, a mill type bit, with an approximately 90° articulation, can be used to mill down the base of the anterior surface of the calcaneus distal to the posterior facet of the calcaneus to a specified depth, which may be just distal the posterior facet. The mill type bit can be guided during cutting by the rails of the first calcaneus cutter guide. Approximately 5 millimeters (mm) of bone may need to be removed from the calcaneus bone surface to make room for the subtalar joint prosthesis through creating a pre-milled trough, although the amount of bone removed may vary depending on patient joint anatomy, etc.

Next, a second calcaneus cutter guide can be fastened to the lateral side of the calcaneus, which may be in a substantially similar location to that of the first calcaneus cutter guide using the pre-milled trough as a guide. After the second calcaneus cutter jig is positioned and set in place, a second cut can be made to the posterior facet surface of the calcaneus with only the second cutter guide installed. The second cutter guide can be then removed.

Both of the calcaneus cuts can be made to specifically conform to the calcaneal facing surfaces of the calcaneal implant as detailed herein, and the calcaneus cuts can also be adjusted to conform to the calcaneal implant, as needed.

The calcaneal and talar implants can be attached to the calcaneus and talus bones respectively, but the attachment of the calcaneal and talar implants can occur in no particular order.

Next, the sheet can be installed between the calcaneal and talar implants. A joint gap is a space between the talar and calcaneal implants. The size of the joint gap could be observed in the undistracted state with the sheet disposed between the talar and calcaneal implants. The surgeon installing the subtalar joint prosthesis can adjust the size of the joint gap, as necessary, by substituting a second sheet of a different thickness to that of the first sheet, if the first sheet does not produce the joint gap of the desired size. A range of thicknesses could be available for the sheet in 1 mm thickness intervals, as an example. There could be larger or smaller size sheets, as necessary, to accommodate a range of sizes in the joint gap.

Finally, the talus and calcaneus bones can be undistracted and the distracter can be removed from the calcaneus and talus bones. The incision can be then closed using standard surgical techniques. The procedure may be complete at this point.

The patient who received the subtalar joint prosthesis may have motion at the subtalar joint, also known as the talocalcaneal joint. The motion of the subtalar joint replacement, or subtalar prosthesis, at the subtalar joint during ambulation, or other activities, may be similar to the kinematics of a healthy native subtalar joint.

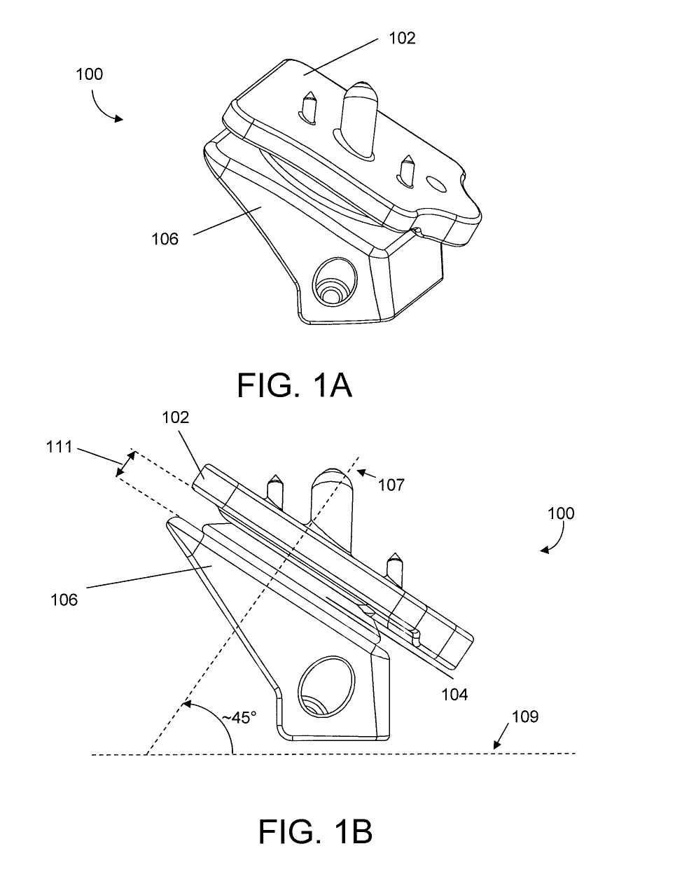

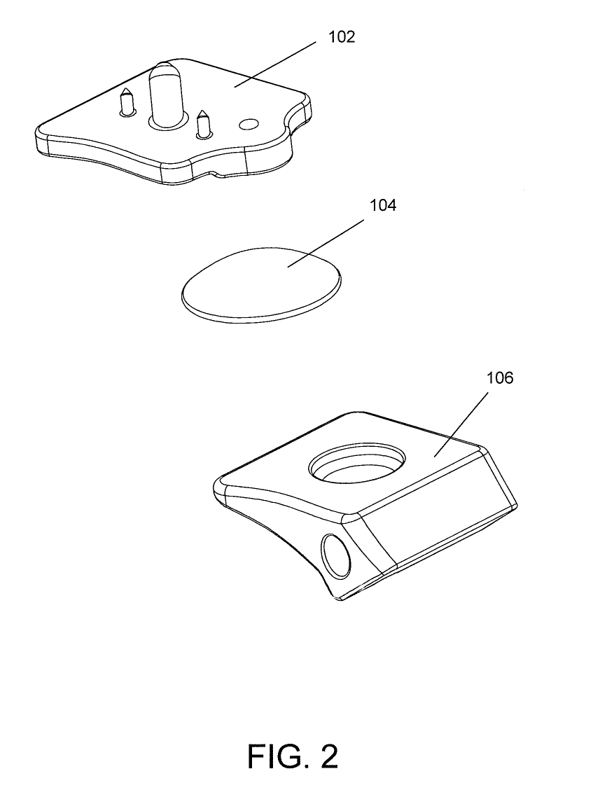

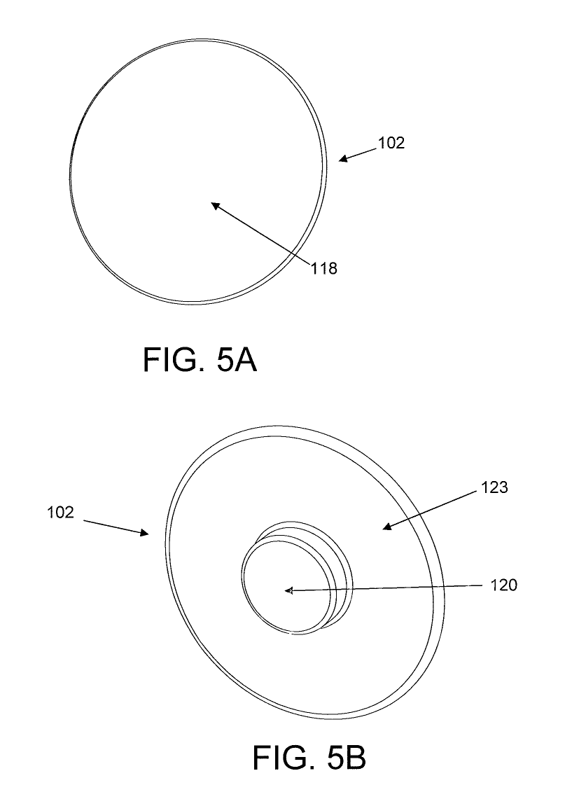

These and other features, procedures, and considerations will be discussed in more detail beginning with a review of FIGS. 1A-B that generally show an exemplary subtalar joint prosthesis 100, in perspective view and side view, respectively. In addition, FIG. 2 shows an exploded view of certain aspects of the subtalar joint prosthesis in accordance with FIGS. 1A-B. The subtalar joint prosthesis 100 may include a talar implant 102, a sheet 104, and a calcaneus implant 106. The sheet 104 is positioned between the talar implant 102 and the calcaneal implant 106. The sheet 104 provides a joint surface 118 (see FIG. 5A) enabling joint motion of the talar implant 102 in relation to the calcaneal implant 106. The talar implant 102 is contactingly adjacent the sheet 104. The sheet 104 is contactingly adjacent the calcaneal implant 106.

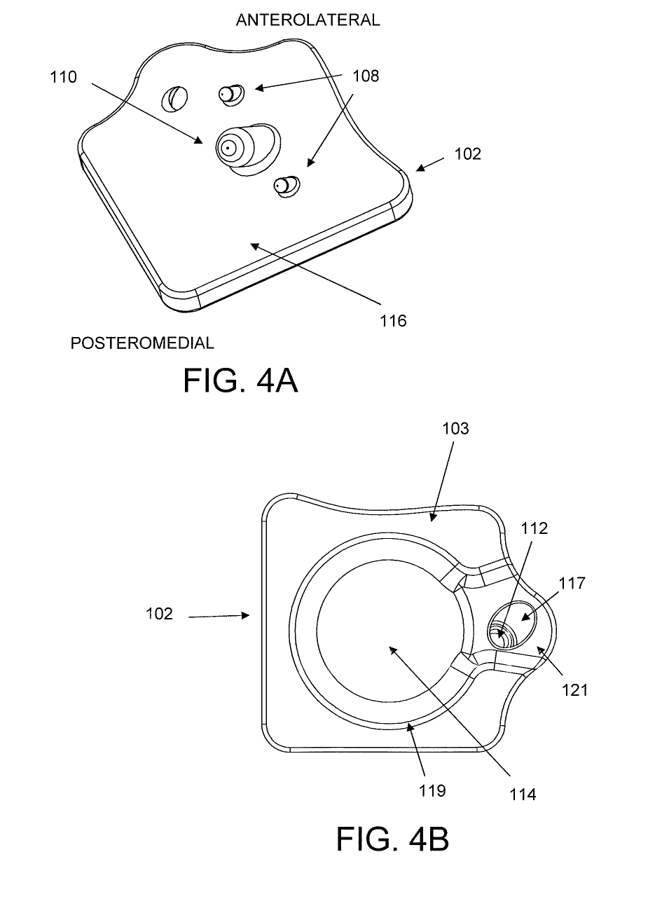

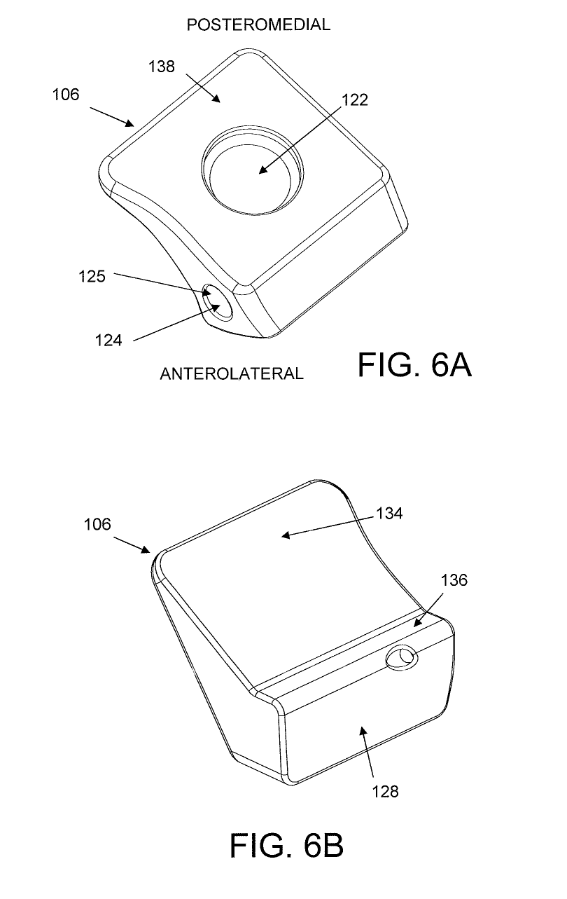

The joint motion of the subtalar joint prosthesis 100 can be similar to the motion of the native subtalar joint that is healthy and functioning normally. FIG. 1B shows that a joint axis 107 of the subtalar joint prosthesis 100 may be situated such that the joint axis can be approximately 45° from the horizontal plane 109, or transverse plane, when a sheet facing surface 103 (see FIG. 4B) of the talar implant 102 is substantially parallel to a sheet facing surface 138 (see FIG. 6A) of the calcaneal implant 106. In this configuration, joint axis 107 is substantially orthogonal to the sheet facing surface 107 and the sheet facing surface 138. This orientation of the joint axis 107 may help the subtalar joint prosthesis 100 enable joint motion similar to the motion of the native subtalar joint in the human, so the subtalar joint prosthesis 100 may have a minimal adverse impact on the movement of the user during motion, such as gait during ambulation.

An implant gap 111 between the talar implant 102 and the calcaneal implant 106 may be dimensioned as needed by inserting the sheet 104 of different sizes. For example, the implant gap 111 may be substantially 3.0 mm.

FIGS. 1A-B also show that the sheet 104 may have relative translation or slide in all directions to the plane substantially parallel to that the sheet facing surface 138 of the calcaneal implant 106, as shown in FIG. 6A, that the sheet 104 rests on when installed or implanted. The combination of rotation and translation motion in the subtalar prosthesis will yield a multi-axis joint that may be able to mimic the functionality of the healthy native subtalar joint. For instance, the sheet 104 may have relative translation in all directions of approximately 2.5 mm, although other dimensions are possible. The amount of relative translation may be restricted by a post 120 (see FIG. 5B) of the sheet 104 interacting with an indentation 122 (see FIG. 6A) of the calcaneal implant 106. The post 120 of the sheet 104 can also promote alignment of the sheet 104 to the calcaneal implant 106 during installation of the subtalar joint prosthesis 100, but the post 120 and the indentation 122 alone or in combination are optional.

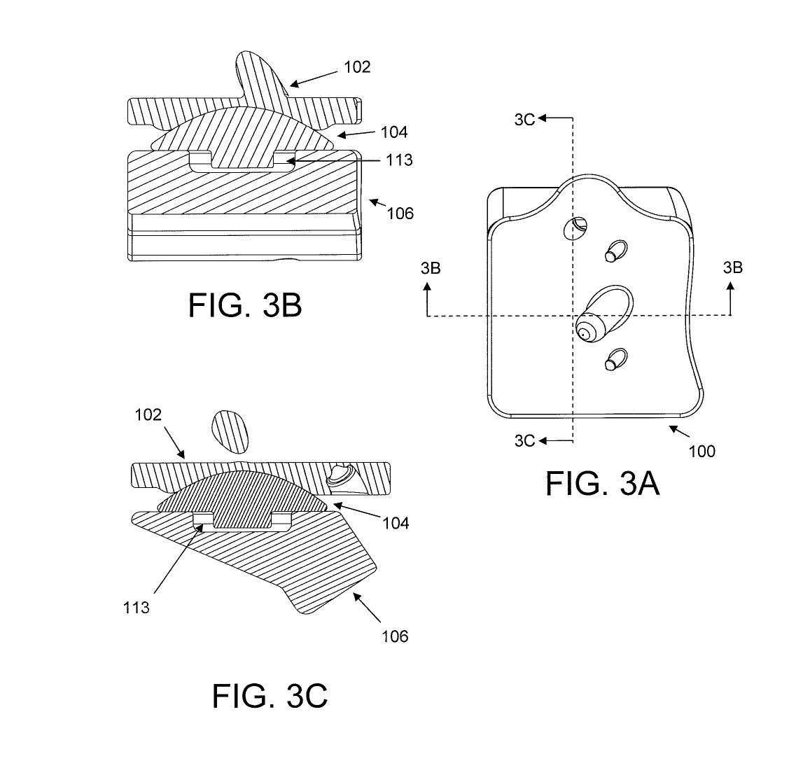

FIGS. 3A-C shows a top plan view of the subtalar joint prosthesis 110 that is assembled and various cross sections of the subtalar joint prosthesis depicting a gap 113 between the post 120 of the sheet 104 and the indentation 122 of the calcaneal implant 106. The post 120 of the sheet 104 and indentation 122 of the calcaneal implant 106 are optional. Without the post 120 and the indentation 122, the amount of relative slide or translation of the sheet 104 in relation to the talar implant 102 and the calcaneal implant 106 could then be restricted by the kinematics of the joint during motion. The native subtalar joint is a tight joint, which may restrict relative motion between the sheet 104 and the talar implant 102 or the calcaneal implant 106 after installation of the subtalar prosthesis. Of course, the interaction of the post 120 and the indentation 122 may restrict translation in one direction while permitting a wider range of motion in another direction, such as restricting motion in the coronal plane and permitting a wider range of motion in the sagittal plane, e.g., through selection of a predetermined shape, predetermined orientation, and predetermined size of both the post 120 and the indentation 122.

If the sheet 104 was fixed relative to the calcaneal implant 106, then the translation of the sheet 104 and talar implant 102 relative to the calcaneal implant 106 could be substantially or completely eliminated. However, translation of the sheet 104 in relation to the calcaneal implant 106 could aid to significantly reduce wear rates of the sheet thereby increasing the lifespan of the subtalar joint prosthesis 100 as well as reducing potential debris generated through use of the subtalar joint prosthesis 100. The translation also could provide an additional degree of freedom further enabling kinematic joint motion more similar to that of the native subtalar joint. The additional degree of freedom also could allow for the talar implant 102 not to be perfectly aligned to the calcaneal implant 106 during installation of the subtalar joint prosthesis 100. This degree of freedom could prove to be a useful feature because accurate and repeatable alignment of the talar to calcaneal implants 102, 106 might be difficult to achieve during installation or use of the subtalar joint prosthesis 100. However, this degree of freedom may not be necessarily needed.

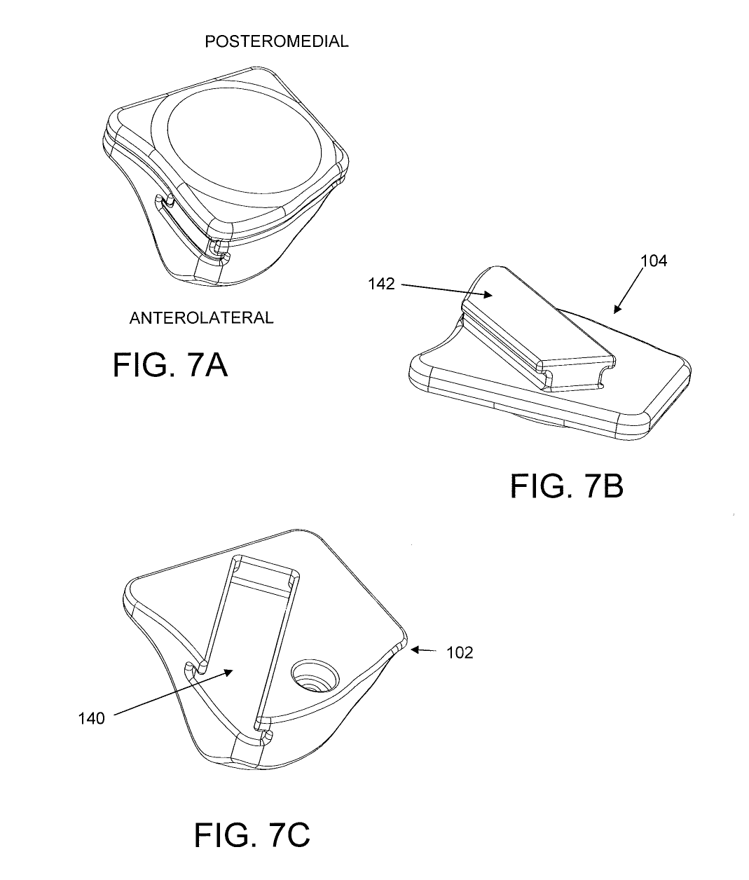

Due to subtalar joint kinematic complexities and unknown subtalar joint prosthesis wear rates, especially for the sheet, the sheet 104 may be fixed relative to the calcaneal implant 106. Fixation of the sheet 104 in relation the calcaneal implant 106 could be accomplished by adding interlocking features to both the calcaneal implant 106 and sheet 104, as shown in FIGS. 7A-C, or by other suitable technique. Fixation of the sheet 104 in relation to the calcaneal implant 106 could also be accomplished by having one body that included some of the functionality of both the calcaneal implant 106 and sheet 104. The sheet 104 could be insert molded or fastened by other geometric or manufacturing techniques to the calcaneal implant 106.

FIGS. 7B-C show a male interlocking connector 142 of the sheet 104 and a female interlocking connector 140 of the calcaneal implant 106. The size, shape, and location of the interlocking connectors may be varied, reversed, etc. depending on need. As shown in FIGS. 7A-C, the interlocking connectors 140, 142 may be positioned approximately 45° from the coronal plane for ease of insertion of the sheet 104 by the surgeon. Anterolateral and posteromedial orientations are shown in FIG. 7A. However, the orientation of the interlocking connectors 140, 142 may not be needed to be 45° from the coronal plane and may be merely an aid during insertion, e.g., insertion of the sheet 104 into the calcaneal implant 106 after the calcaneal implant 106 is affixed to the calcaneus. The angle and orientation of the interlocking connectors 140, 142 could be adjusted to be more or less than approximately 45° depending on need. The interlocking connectors 140, 142 could, but are not needed to be, as large as possible, which might increase subtalar joint prosthesis 100 and interlocking connectors 140, 142 integrity through increased surface area so that the subtalar joint prosthesis 100 lasts longer and is less prone to failure. The sheet 104 could also be fastened to the calcaneal implant 106 by biocompatible epoxy or other suitable fastener. If the sheet to implant orientation is reversed, the sheet 104 could be fastened to the talar plate 102 to substantially reduce or substantially eliminate relative motion between the sheet 104 and the talar plate 102.

FIGS. 4A-B shows exemplary views of the talar implant 102. Anterolateral and posteromedial orientations are shown in FIG. 4A. The thickness between the talus facing surface 116 and the sheet facing surface 103 may be 3.0 mm, but other dimensions are possible. The talar implant 102 may be approximately 1.0 mm thickness at its thinnest location and approximately 4.0 mm at its thickest location. Again, other dimensions are possible.

The talar implant 102 may have staking pins 108 which may be used to hold the talar implant 102 in place prior to fixation of the talar implant 102 to the talus bone with a fastener. The number, size, shape, orientation, and location of the staking pins 108 may be varied or the staking pins may be completely eliminated based on need. The talar implant 102 also may have a post 110 which may help promote long term fixation of the talar implant 102 to the talus bone. Two or more staking pins 108 and the post 110 may restrict rotation of the talar implant 102 with respect to the talus.

The staking pins 108, the post 110, and a talus facing surface 116 of the talar implant 102 may all be coated with a porous media applied via plasma spray or similar and/or different application process depending on need. The porous media may enable bony ingrowth of the talus bone to the talar implant 102 of the subtalar joint prosthesis to promote long term fixation and stability of the subtalar joint prosthesis 100. The talar and calcaneal implants 102, 106 may be made of any suitable biocompatible metal, metal alloy, ceramic, or other suitable material.

The staking pins 108 and the post 110 for bone ingrowth may be positioned for interaction with the talus on insertion of the subtalar joint prosthesis 100. The location of the subtalar joint prosthesis 100 may vary; however, the joint axis 107 (see FIG. 1B) of the subtalar joint prosthesis 100 may be positioned in a similar orientation to that of the native subtalar joint axis to improve restoration of proper joint kinematics during gait or other motion. The staking pins 108 and the post 110 for bone ingrowth may be dimensioned as needed with either the staking pin 108 or the post 110 being larger than the other or the staking pin 108 and the post 110 being substantially the same size, as needed. Additionally, any number of the staking pins 108 and the post 110 for bone ingrowth may be provided on the talar implant 102 as necessary.

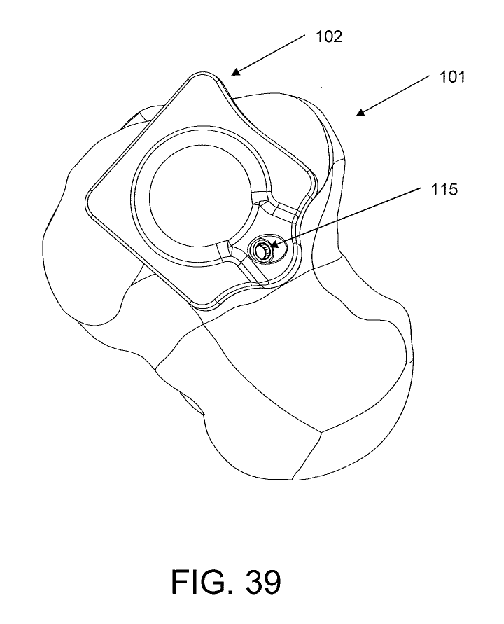

Short term fixation of the talar implant 102 may come from a talus fastener 115 (see FIG. 39), such as a screw, stud, bolt, nail, etc., which can be inserted through a talus fastener aperture 112 of the talar implant 102 after the talar implant has been tapped in place by the surgeon driving the staking pins 108 into the talus bone. The talus fastener aperture may have a diameter of approximately 2.5 mm and a recess diameter of approximately 4.0 mm, but other dimensions are possible.

Bone cement could also be used to anchor/attach the talar implant 102 to the talus. Exemplary bone cement is the Simplex P Bone Cement family of products from Stryker Corporation, 2825 Airview Boulevard, Kalamazoo, Mich. 49002 U.S.A., but other types of bone cement could be used. Bone cement may be designed to fill the free space between the prosthesis and the bone, and the bone cement can also add an elastic zone. This elastic zone may help the bone cement better absorb the forces imparted during motion which may help the talar implant 102 remain in place long term although this might not be necessary. The talus fastener 115 might allow for short term fixation of the talar implant 102 to the talus before bony ingrowth to the talar implant 102 can help promote long term fixation. The bony ingrowth may be aided by the presence of the porous type media applied to the staking pins 108, the post 110, the talus facing surface 116, etc. as previously described. Accordingly, bone cement could be used in conjunction with the talus fastener 115.

The talus fastener aperture 112 may also include a recessed area 117 for the head of the talus fastener 115 to reside in after installation of the talus fastener 115. Recessing the talus fastener 115 in this manner may prevent potential interference between the head of the talus fastener 115 with the sheet facing surface 138 of the calcaneal implant 106 (see FIG. 6A) or the sheet 104 (see FIG. 1), during installation of the subtalar joint prosthesis or after installation of the subtalar joint prosthesis during joint motion.

The talus fastener aperture 112 may be most conveniently disposed on the lateral or anterolateral aspect of the talar implant 102 for ease of insertion of the talus fastener 115 through the talus fastener aperture 112 into the talus. Debris from installation or use of the subtalar joint prosthesis 100 may come from any part of the subtalar joint prosthesis 100, such as the talar implant 102, the sheet 104, the calcaneal implant 106, etc., which may cause undesirable outcomes, such as possibly causing inflammation. The sheet 104 may be the part most likely to produce debris, or produce the most debris. If the talus fastener aperture 112 is positioned to intersect with the sheet facing surface 103 of the talar implant 102, the talar implant may produce more debris than if the talus fastener aperture 112 does not intersect with the sheet facing surface 103, such as by the talus fastener aperture 112 being disposed on the lateral or anterolateral aspect of the talar implant 102. Correct, may be need clarification? Similar debris production issues may apply to a fastener aperture intersecting with the sheet facing surface 138 of the calcaneal implant 106 (see FIGS. 6A-B).

The talus fastener aperture 112 of the talar implant 102 interacting with the talus fastener 115 may perform several functions. The talus fastener aperture 112 and the talus fastener 115 interaction may help stabilize the talar implant 102 to the talus, especially in the short term to provide initial stability of the talar implant 102 with respect to the talus until bony ingrowth into the talar implant 102 provides effective long-term stability. The talus fastener aperture 112 and the talus fastener 115 interaction may prevent relative translation or rotation of the talar implant 102 to the talus, which may be especially useful if only one staking pin 108 or post 110 is provided by the talar implant 102.

The staking pins 108, the post 110, and the talus fastener aperture 112 can all be angled approximately 45° from both the sagittal and transverse planes for ease of installation by the surgeon of the talar implant 102 and the fastener 112. The staking pins 108 and the post 110 can be located at approximately 45° from both the sagittal and transverse planes otherwise the staking pins 108 and the post 110 might be more likely to impinge insertion of the talar implant. During the surgical procedure, the subtalar joint will typically be opened in the lateral aspect and the talar implant 102 will be aimed medially for insertion by the surgeon.

These angles can be varied in magnitude and direction based on need, and approximately 45° angles are not necessarily needed. For example, the angles of the staking pins 108, the post 110, and the talus fastener aperture 112 could be set to be substantially perpendicular to the talus facing surface 116 of the talar implant 102. However, having the axis orientation of each of the staking pins 108, the post 110, and the talus fastener aperture 112 substantially perpendicular to that of the talus facing surface 116 of the talar implant 102 could increase installation difficulty of the talar implant 102 and the talus fastener 115 by the surgeon. Of course, the staking pins 108, the post 110, and the talus fastener aperture 112 may be aligned with substantially the same angles as each other with regards to the talar implant 102. The staking pins 108 and the post 110 may be substantially parallel to each other, which may ease installation of the talar implant 102. The talus fastener aperture 112 may have a substantially different angle in relation to the talar implant 102 than the angle of the staking pins 108 or the post 110 in relation to the talar implant.

The talar implant 102 may also include a female joint surface 114 which interfaces directly with the male joint surface 118 of the sheet 104 (see FIG. 5A). An exemplary female joint surface 114 has surface area of 205 mm², but other dimensions are possible. Both surfaces 114, 118 may be curvilinear in shape. The joint surfaces 114, 118 may be provided in a variety of sizes which may be used to set predetermined limits or range of motion of the sheet 104 in relation to the talar implant 102. For example, tilt in the sagittal plane may yield a range of motion of approximately 20° for the subtalar prosthesis with approximately 10° coming from flexion starting at the neutral position as shown in FIG. 1B and approximately 10° coming from extension starting at the neutral position as shown in FIG. 1B. An increase in surface area for the joint surfaces 114, 118 interacting in the subtalar joint prosthesis may reduce the load per square area, which may reduce wear rates. A decrease in surface area for the joint surfaces 114, 118 interacting in the subtalar joint prosthesis may increase the load per square area, which may increase wear rates. The male-female orientation of the joint surfaces 114, 118 may be reversed. However, this reversal in orientation could substantially reduce the thickness of the sheet 104, which might reduce the useful life of the subtalar joint prosthesis.

The joint surface 114 may be surrounded by a rim 119. The rim 119 may increase the gap or clearance between the sheet facing surface 103 of the talar implant 102 and the sheet facing surface 138 of the calcaneal implant 106 for increased range of motion of the subtalar joint prosthesis 110 after installation. The rim 119 may provide 1-2 mm, or more or less, material thickness to compensate for thickness of the recessed area 117, otherwise, the recessed area 117 may create a localized area of impingement with the sheet 104 or the calcaneal implant 106.

The talus fastener aperture 112 may be surrounded by an aperture rim 121 which may allow a longer talus fastener 115 or head of the talus fastener 115. The rim 119 and the aperture rim 121 are each optional and either the rim 119 or the aperture rim 121 may be present without the other being present. The thickness of the rim 119 or the aperture rim 121 may be adjusted as needed to promote or restrict motion of the subtalar joint prosthesis after installation. However, the thickness of the talar implant 102, the sheet 104, and the calcaneal implant 106 are generally selected to not be thick in order to allow range of motion in the subtalar joint prosthesis 100 without significant impingement during motion. However, a full range of motion in the subtalar joint prosthesis 100 may be limited only by the biomechanics of the body in which the subtalar joint prosthesis 100 has been inserted. In other words, the user of the subtalar joint prosthesis may have substantially normal range of motion at the subtalar joint in the subtalar prosthesis.

FIGS. 5A-B show exemplary views of the sheet 104. The sheet 104 may comprise the male joint surface 118 and the male post 120 on a calcaneal implant facing surface 123. The male joint surface 118 may have a surface area of approximately 370 mm, but other dimensions are possible. The calcaneal implant facing surface may have a surface area of approximately 260 mm², but other dimensions are possible. The sheet 104 may be approximately 1.0 mm thickness at its thinnest location and approximately 7.0 mm at its thickest location.

In addition to the design shown in FIGS. 5A-B, other designs are within the scope of the present disclosure. For example, the lateral margins of the post 120 may be substantially congruent with the perimeter of the male joint surface 118. As another example, the sheet 104 may provide a flange that extends beyond the perimeter of the male joint surface 118. The flange may be a substantially planar surface. The flange may also extend beyond the perimeter of the male joint surface 118, or the flange may not extend beyond the perimeter of the male joint surface 118. These examples are exemplary and not limiting.

The joint surface 118 may be a substantially curvilinear surface to promote rotation motion. The male post 120 could be made smaller or larger in diameter which could either increase or decrease relative translation of the sheet 104, respectively, relative to the calcaneal implant 106 for a fixed size of the indentation 122 (see FIG. 6A) of the calcaneal implant 106. The male post 120 may be 2.0 mm in height and 7.0 mm in diameter or width, but other dimensions are possible.

The post 120 is shown as a cylinder, which may provide the most freedom of translation in the substantially two-dimensional plane. However, the post 120 may have any shape and that shape may be used to restrict or favor translation of the sheet 104 in relation to the talar implant 102 or the calcaneal implant 106. Alternatively, the sheet 104 could also be designed to have the indentation, and the calcaneal implant 106 could also be designed to have the post. However, this orientation could substantially reduce the thickness of the sheet 104, which might reduce the useful life of the subtalar joint prosthesis.

The sheet 104 may provide a curvilinear surface that interacts with a curvilinear surface provided by the calcaneal implant 106, and the talar implant 106 and the sheet 104 may provide the indentation and post interaction. All of the curvilinear surfaces may be continuously curvilinear.

The sheet 104 could be made out of a polymer based material or other material which may not be as hard as that of the talar and calcaneal implants 102, 106. The sheet 104 could be designed to wear at a greater, or faster, rate than the wear rate of the talar and calcaneal implants 102, 106. This wear rate difference between the sheet 104 and the talar and calcaneal implants 102, 106 might lead to a situation where the sheet 104 wears out, and the sheet 104 can be more easily replaced as opposed to needing to replace the talar or calcaneal implants 102, 106, which might need a substantially more difficult surgical procedure for replacement.

FIGS. 6A-B show exemplary views of the calcaneal implant 106. Anterolateral and posteromedial orientations are shown in FIG. 6A. The calcaneal implant 106 may include the female indentation 122 for the post 120 (see FIG. 5B) of the sheet 104 to be inserted into. The diameter of the indentation 122 could be made smaller or larger in diameter which could either decrease or increase relative translation of the sheet 104 relative to the calcaneal implant 106, respectively. The indentation 122 may be 2.5 mm in depth and 12.0 mm in diameter, but other dimensions are possible. The indentation 122 is shown as a circle, but the indentation 122 can be any shape, such as oval, triangular, etc., for interaction with the post that can be of any shape also. The indentation 122 and the post 120 interaction may be used to promote or inhibit motion in certain directions. The calcaneal implant 106 could also include a male post as opposed to a female indentation, and the sheet 104 could also include a female indentation as opposed to a male post.

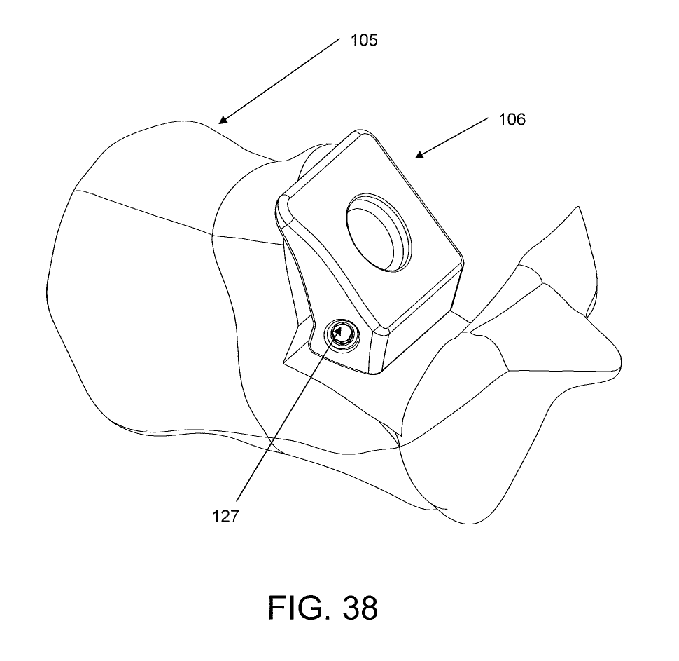

Once the calcaneal implant 106 is installed in the calcaneus bone and tapped in place into the calcaneus bone, a calcaneus fastener 127 (see FIG. 38) may be installed through the calcaneus fastener aperture 124 of the calcaneal implant 106 to provide short term fixation of the calcaneal implant 106 to the calcaneus. Bone cement could also be used to anchor/attach the calcaneal implant 106 to the calcaneus. Bone cement may be designed to fill the free space between the prosthesis and the bone, and the bone cement can also add an elastic zone. This elastic zone may help the bone cement better absorb the forces imparted during motion which may help the calcaneal implant 106 remain in place long term, although this might not be necessary. Accordingly, bone cement could be used in conjunction with the calcaneus fastener 127.

The surfaces 128, 134, and 136 of the calcaneal implant 106 may all be coated with a porous media applied via plasma spray or similar and/or different application process depending on need. The porous media may enable bony ingrowth of the calcaneus bone into the calcaneal implant 106 allowing for long term fixation of the subtalar joint prosthesis due to the presence of the porous type media, previously described. In other words, any of the surfaces of the subtalar joint prosthesis may be coated, or made of any suitable material, so as to promote bony ingrowth and long-term fixation of the subtalar joint prosthesis as needed.

The calcaneus fastener aperture 124 may also include a recessed area 125 for the head of the calcaneus fastener 127 to reside after installation of the calcaneus fastener 127 through the calcaneus fastener aperture 124, but the recessed area 125 is not particularly necessary and could be considered optional. The diameter of the calcaneus aperture 124 may be approximately 2.0 mm and the diameter of the recessed area 125 may be approximately 4.5 mm, but other dimensions are possible. The calcaneus fastener aperture 124 may be positioned immediately below the sheet facing surface 138. The sheet facing surface may have a surface area of approximately 500 mm², but other dimensions are possible. The calcaneus fastener aperture 124 may be also angled approximately 20° from the transverse plane for ease of installation of the fastener in the calcaneus by the surgeon. The angular offset may be varied in magnitude and direction based on need and may not be necessarily needed to be a particular magnitude and direction. The size, location, geometry, and number of the calcaneus fastener aperture 124 may be varied depending on need. As with the talar implant 102, the calcaneal implant 106 the fastener apertures positioned and varied in number to optimize interaction with the bone that in the case of the calcaneal implant is the calcaneus bone.

It should be understood that the subtalar joint prosthesis 100 including all parts, or subcomponents, of the subtalar joint prosthesis 100 could be made scalable to be larger or smaller to conform to variable joint and bone sizes and anatomies from patient to patient. A larger or smaller talar implant maybe needed in conjunction with a smaller or larger calcaneal implant or any combination of these. The talar implant 102, the sheet 104, and the calcaneal implant 106 can be custom fit and dimensioned for installation in the patient.

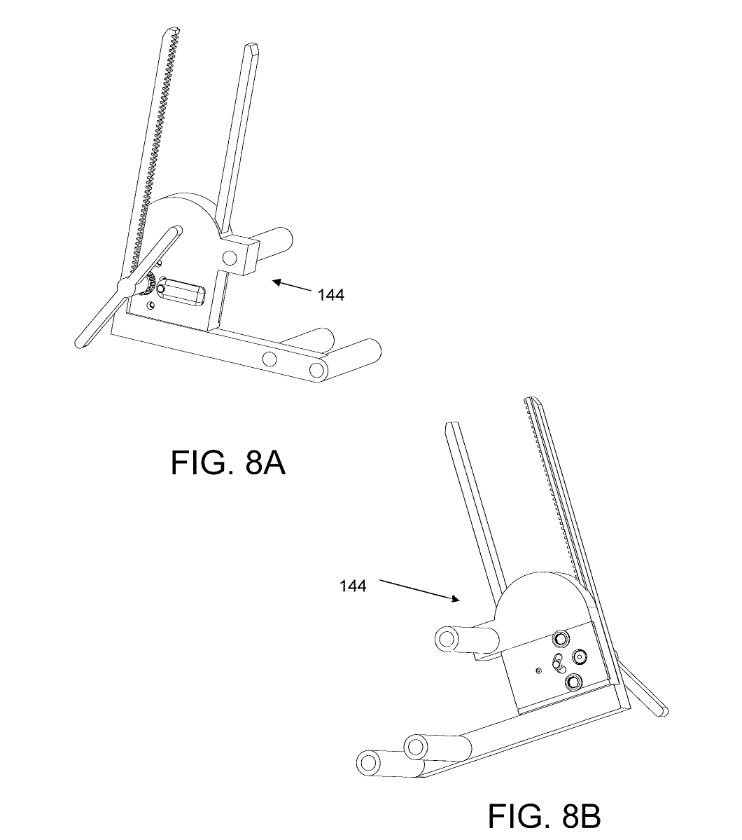

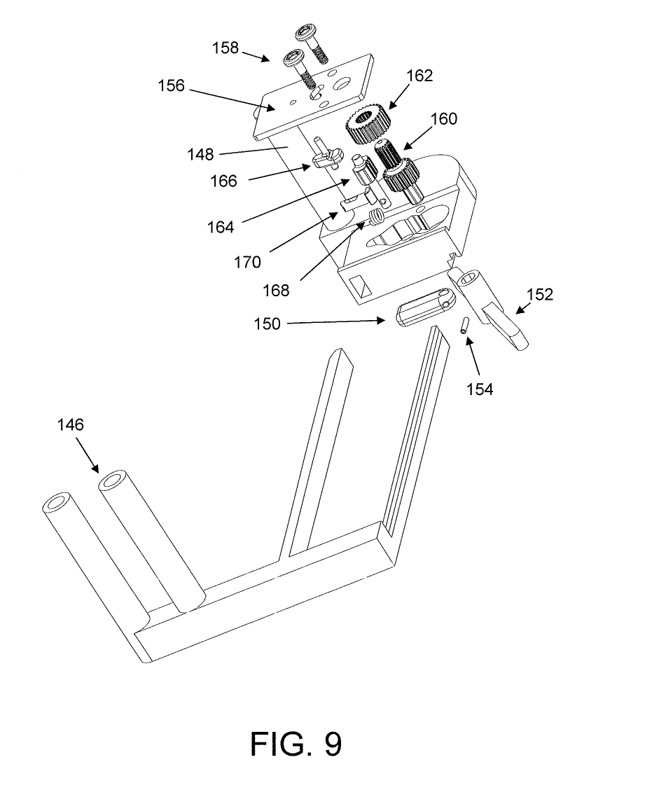

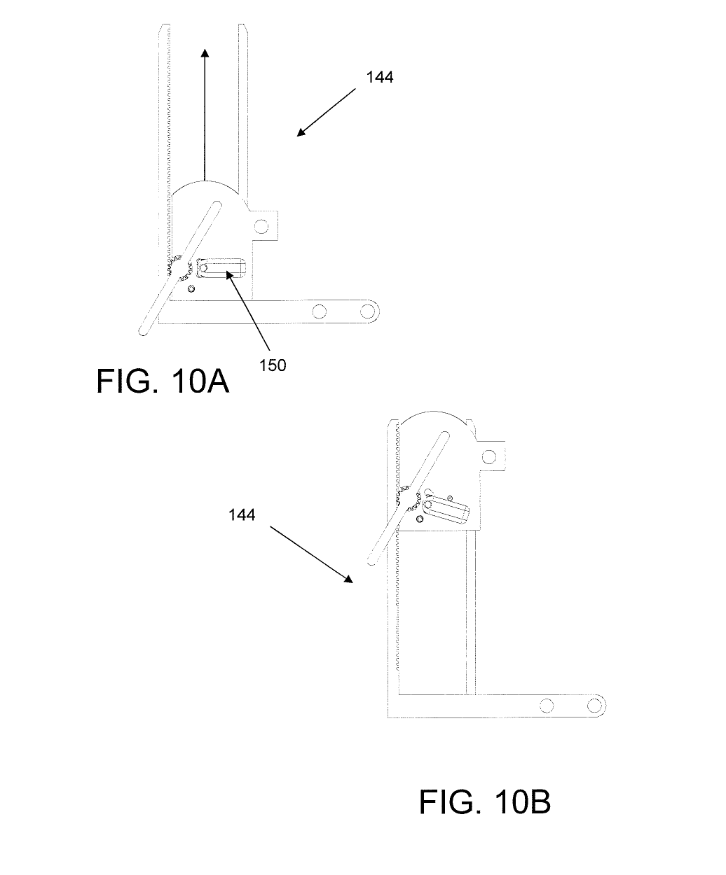

FIGS. 8A-B show exemplary views of a distracter 144. The distracter 144 can be attached to the talus and calcaneus bones using fasteners, such as screws, nails, etc. FIGS. 9A-B show an exploded view of the distracter 144 may comprise 12 main components as shown in FIGS. 8A-B, although more or fewer components may be used as needed. Each component will be described in more detail herein and shown in FIGS. 10-22.

The distracter 144 may be positioned in such a way after attachment to the calcaneus and talus bones to not interfere with the surgical procedure or tools used to install the subtalar joint prosthesis 100 described herein in FIGS. 10-22. Due to the length of a fastener aperture(s) 188 of the distracter front body 146 and a fastener aperture 194 of a distracter main body 148, the distracter 144 may remain a suitable distance away from the foot in the sagittal plane when attached to the calcaneus and talus bones, respectively.

After the distracter 144 is attached to the talus and calcaneus bones, the button 150 may be pushed to a first position, such as the up position, to activate the wrench type mechanism, and the handle 152 may be turned in a first rotation direction, such as the counterclockwise direction, to extend the distracter to a predetermined distance of distraction. The distracter 144 might extend approximately two inches, although the distance of extension can be varied to be more or less based on need, in order to facilitate insertion of the subtalar joint prosthesis by providing enough room or clearance for insertion of the subtalar joint prosthesis.

When the handle 152 is rotated the main gear 160 can also be rotated. This rotation may be provided by interaction of the male portion of the wrench head 224 of the main gear and female portion of the wrench head 256 of the handle 152. As the main gear 160 is rotated, the gear type teeth 222 may be engaged with the gear type teeth 182 of the distracter front body 146. This wrench type mechanism could allow the distracter main body 148 with subcomponents, to move in a first direction, such as away from that of the distracter front body 146, wherein distraction is shown to correspond to the direction of the arrow.

A guide rail 186 of the distracter front body 146 may slide through a guide post slot 204 of the distracter main body 148 during distraction of the talus bone from the calcaneus bone, which may promote stability of the distracter 146. Additional stability of the distracter 146 may be promoted during distraction by the female slot 206 of the distracter main body 148 coupled with the male guide 184 of the distracter front body. The features described herein which promote stability may be varied in size, shape, geometry, and location and also may be eliminated based on need.

The button 150 can be pushed in a second position, such as the down position, to activate the wrench type mechanism such that the distracter could be retracted in the reverse direction. The button could also be set in a third position, such as the neutral position, to allow freedom of travel either to extend or retract the distracter as necessary.

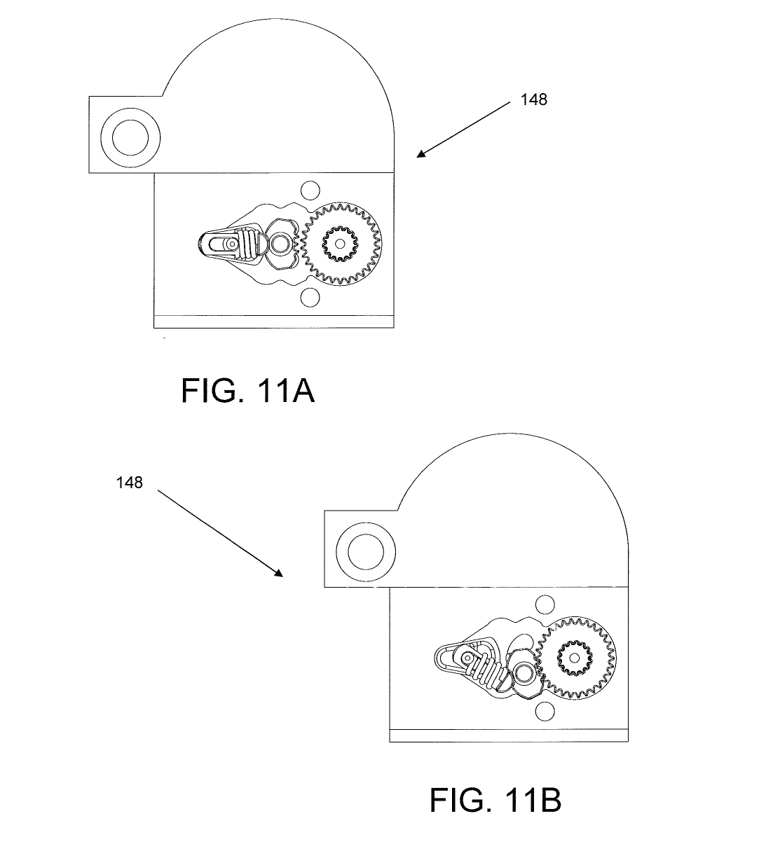

FIGS. 11A-B show a back view of the distracter subassembly that highlights the components that make up the wrench type mechanism. This wrench type mechanism will not be described in great detail as the mechanism can be similar to the mechanism found in U.S. Pat. No. 6,904,832, which is totally incorporated herein by reference.

When the button 150 is activated in the up and/or down positions from the neutral position, the button may force the guide pin 230 of the locking wrench gear 164 to travel along the path of the guide post aperture 198 of the distracter main body 148 as well as the guide post aperture 214 of the cover plate 156. This positioning could enable the gear type teeth 232 of the wrench gear 164 to come into contact with the gear type teeth 226 of the wrench gear 162.

Once the gear type teeth 226 can be engaged, they can lock the mechanism in a fixed state allowing for distraction, either extension or retraction, to be achieved by turning the handle 152 in the counterclockwise or clockwise directions, respectively. The mechanism could be designed in a similar fashion allowing for counterclockwise direction of the handle 152 providing retraction, but may not be necessarily needed.

As the handle 152 is rotated, the main gear 160 also can be rotated which can also rotate the wrench gear 162. The main gear 160 may be rotationally fixed to the wrench gear 162. This fixation may be provided by the female spline type 220 of the main gear 160 as well as the male spline type 228 of the wrench gear 162. Although a spline type can be used to provide fixation, this fixation could be achieved in many different ways and still stay within the scope of the disclosure. As the wrench gear 162 is rotated, the gear type teeth 226 can advance past the gear type teeth 232 of the locking wrench gear 164.

Advancement of the gear type teeth 226 can be provided by the bias element pin 170 and bias element 168. The bias element 168 can be a spring or other suitable biasing member. The bias element pin 170 can be translated in a direction away from the gear type teeth interface. This translation can be provided by the slotted aperture 248 of the bias element pin 170. The pin body 238 of the pin guide 166 can be fixed in place relative to the distracter main body 148. This fixation can be provided by the pin body 238 of the pin guide 166 as the top portion could be inserted into the pin aperture 216 of the cover plate 156 and bottom portion into the aperture 196 of the distracter main body 148.

The pin guide 166 can remain fixed in place while the bias element pin 170 translates away from the gear type teeth interface. The bias element 168 could allow the gear type teeth 226 to remain engaged fixing the distracter 144 in place once a proper distraction distance can be achieved.

There may be a detent 208 of the distracter main body 148 that the guide pin 230 of the locking wrench gear 164 rests in. This may help the button 150 remain in the neutral position prior to activation. Although this same effect may be achieved in many different ways depending on need.

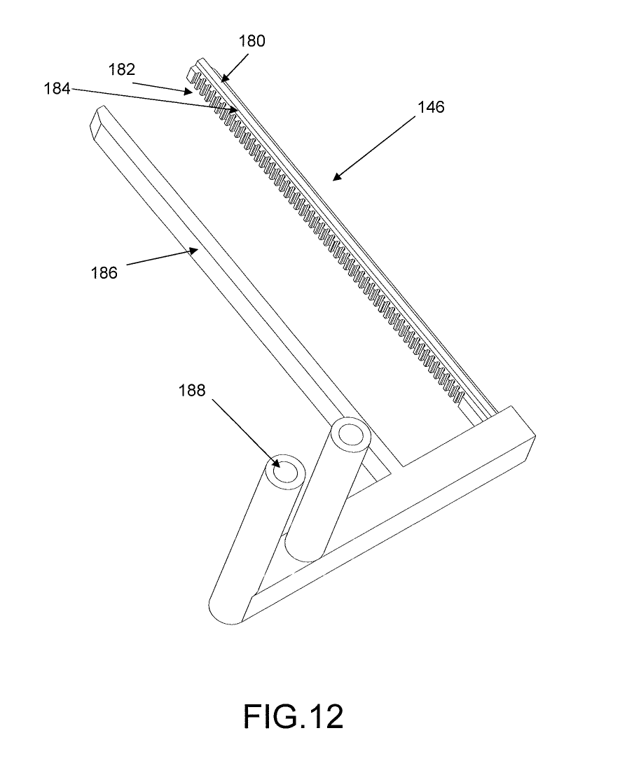

FIG. 12 shows an exemplary view of the distracter front body 146. All of the features of the distracter front body 146 have already been described herein except for the fastener aperture 188. The fastener aperture 188 can be for fasteners, such as screws, nails, bolts, etc. that may be used to attach the distracter front body 146 to the calcaneus bone. The size, location, geometry, and number of aperture 188 maybe varied based on need to attach the distracter front body 146 to the calcaneus bone.

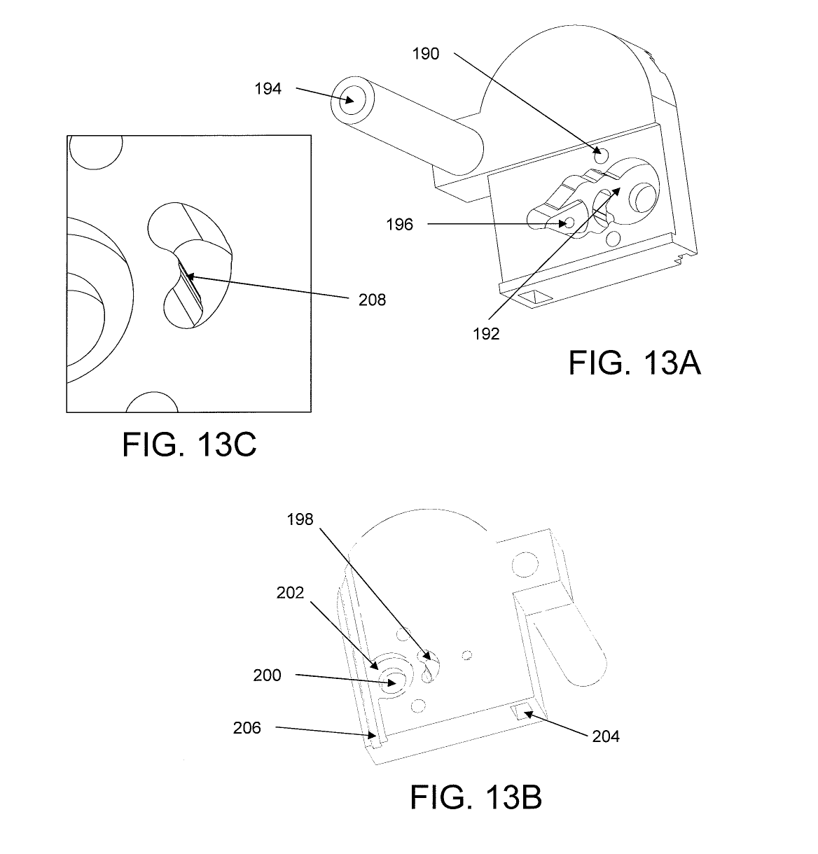

FIGS. 13A-C show exemplary views of the distracter main body 148. Many of the features have already been described herein. There can be a recess 202 which may make room for the gear type teeth 222 of the main gear 160. This recess 202 could be eliminated based on need, which might expose more of the gear type teeth 222 of the main gear 160. The aperture 200 may provide clearance for the splined shaft 220 of the main gear 160. Apertures 190 may provide fixation for fasteners, such as screws, nails, etc. that may be used to attach the cover plate 156 to the distracter main body 148. Aperture 194 may provide for a fastener used to attach the distracter main body 148 to the talus bone. The size, location, geometry, and number of the aperture 194 maybe varied based on need.

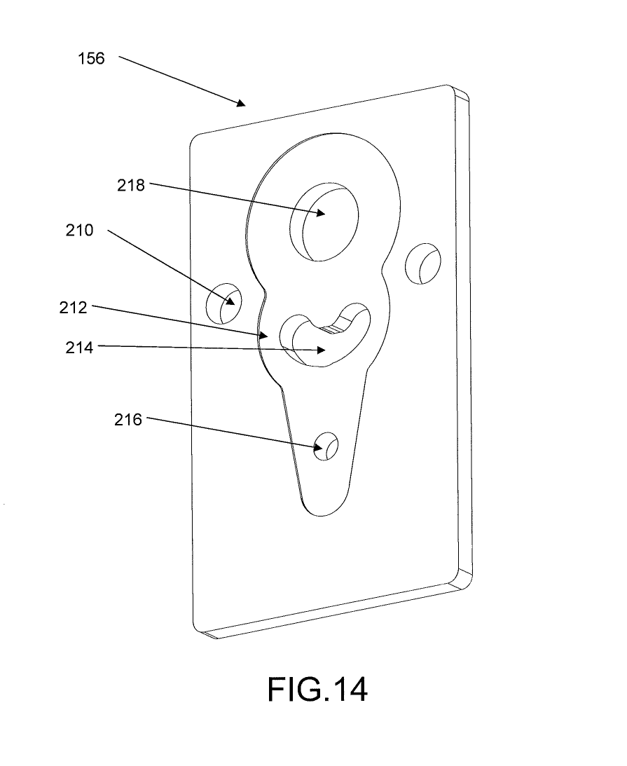

FIG. 14 shows an exemplary view of the cover plate 156. Many of the features have already been described herein. Surface 212 may have a slight recessed surface that provides room for the components which make up the wrench type mechanism. This recess could be eliminated by simply making the subtalar joint prosthesis thicker.

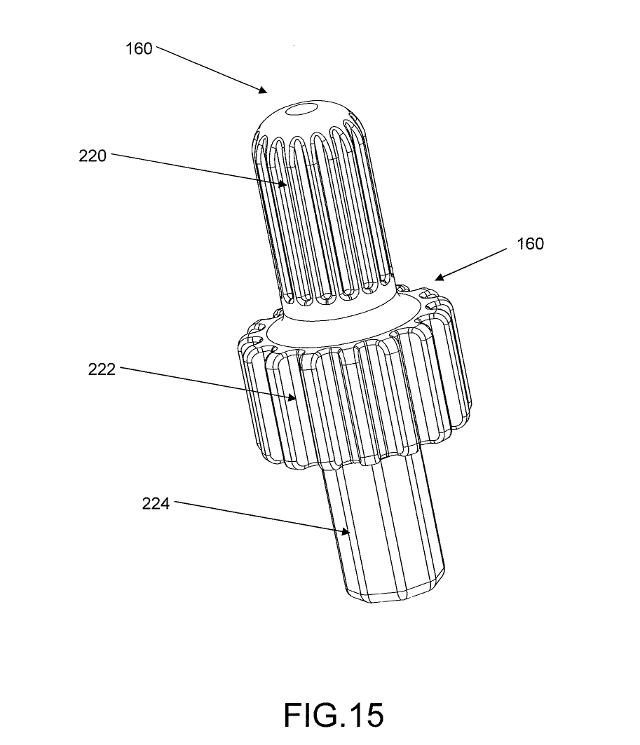

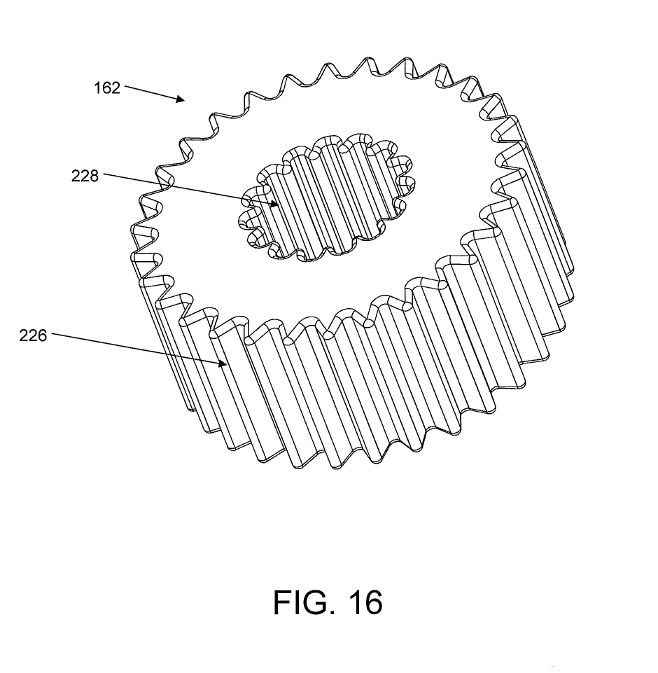

FIGS. 15-16 show perspective use of the main gear 160 and wrench gear 162, respectively. Many of the features, shown in FIGS. 15-16, have been previously described herein. The male wrench head 224 may provide fixation to the female wrench aperture 256. Any type of handle or tool could be attached to the male wrench head 224 of the main gear 160 to provide distraction (extension or retraction) through rotation of the handle or tool and therefore rotation of the main gear 160.

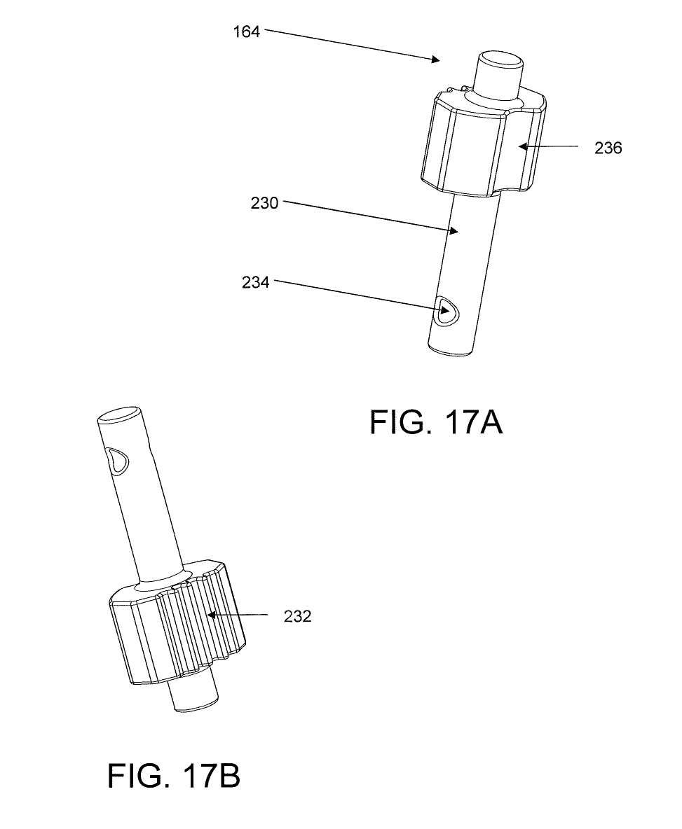

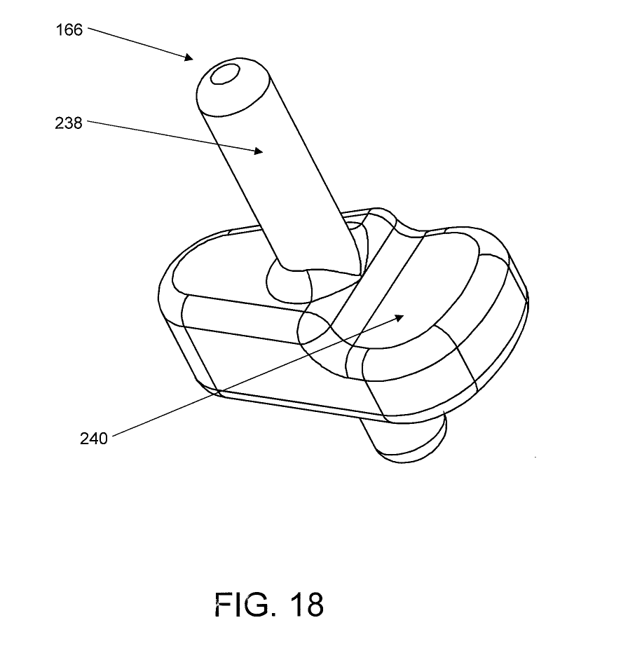

FIGS. 17A-B and FIG. 18 shows an exemplary view of both the locking wrench gear 164 and pin guide 166, respectively. Many of the features, shown in these figures, have already been described herein. The aperture 234 of the locking wrench gear 164 could allow for the button pin 154 to be inserted within. This provides fixation of the button 150 to the locking wrench gear 164. However, there could be many ways to provide fixation of the button for activation of the wrench mechanism and still stay within the scope of the disclosure. The recessed surface 240 of the pin guide 166 may provide clearance for the bias element 168 to reside within.

The gear teeth 182 of the distracter front body 146, the gear teeth 222 of the main gear 160, the wrench gear teeth 226 of the wrench gear 162, the wrench gear teeth 222 of the guide pin 230, and other aspects of the present disclosure may all be varied in size, shape, and number depending on need. The set of mating teeth of each component conform to another set of mating teeth of each component. For example, changing the size, shape, and number of the gear teeth 182 of the distracter front body 146 and the gear teeth 222 of the main gear 160 could also be done to achieve a different gear ratio based on need.

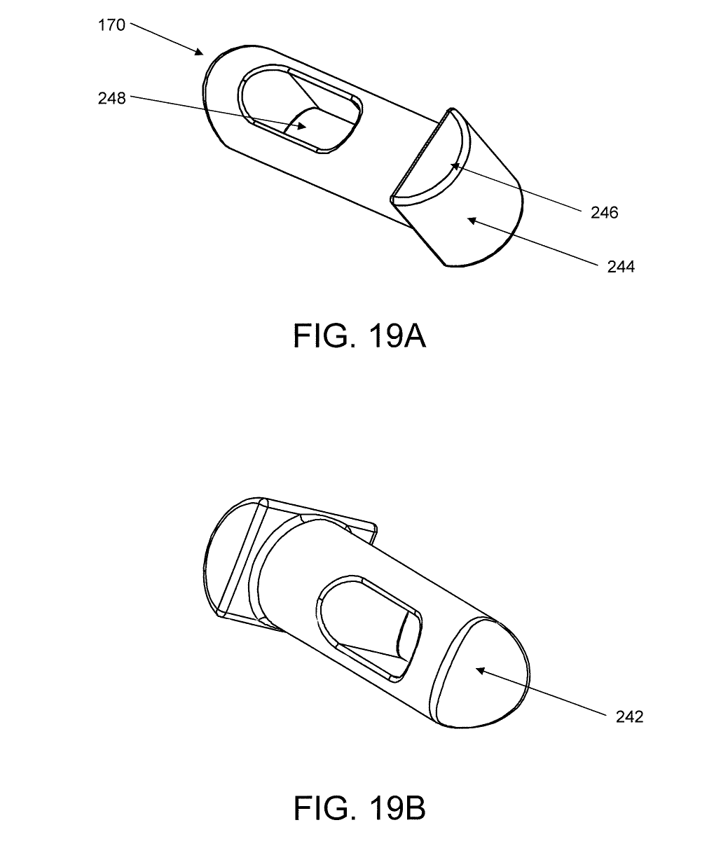

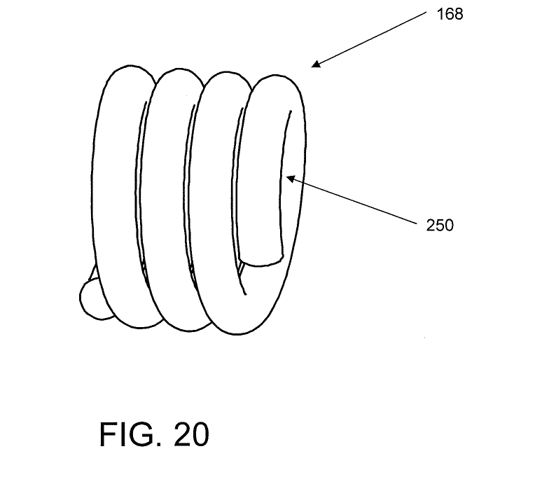

FIGS. 19A-B show perspective use of both the bias element pin 170. The bias element pin 170 has a first surface 248, a second surface 244, a pin head 246, a slotted aperture 248, and a body 249. FIG. 20 shows a side view of the bias element 168. When the distracter is assembled, the body 249 of the bias element pin 178 is located within the bias element 168.

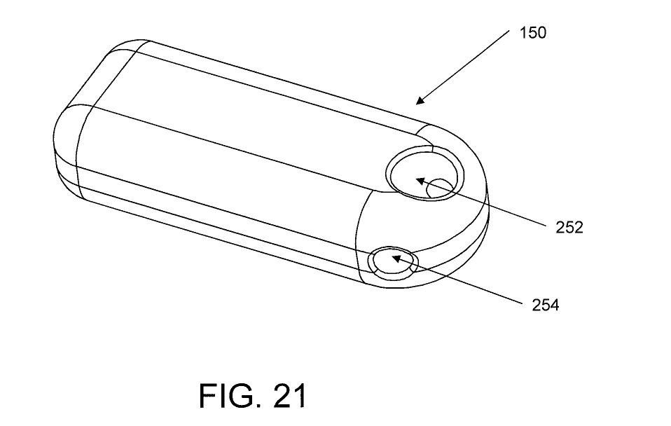

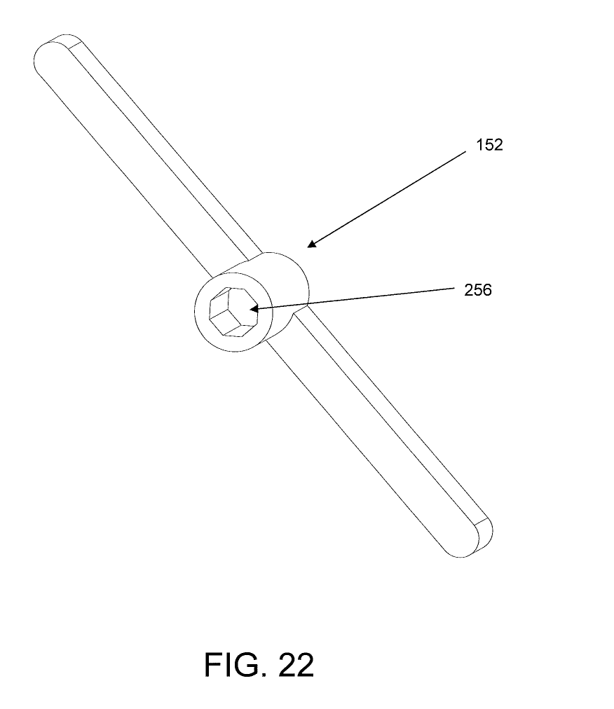

FIGS. 21-22 show a perspective view of the button 150 and handle 152, respectively. Many of the features have been previously described herein. Aperture 252 of the button 150 which may provide an opening for the pin 230 of the locking wrench gear 164 to be inserted.

All of the features and components of the distracter 144 may be varied in size, shape, location, and number depending on need and application and stay within the scope of the disclosure.

Due to the plethora of possible ways of constructing a distracter, many of the components and features which comprise the distracter 144, as described herein, could be modified in size, shape, location, and number depending on need and stay within the scope of the disclosure.

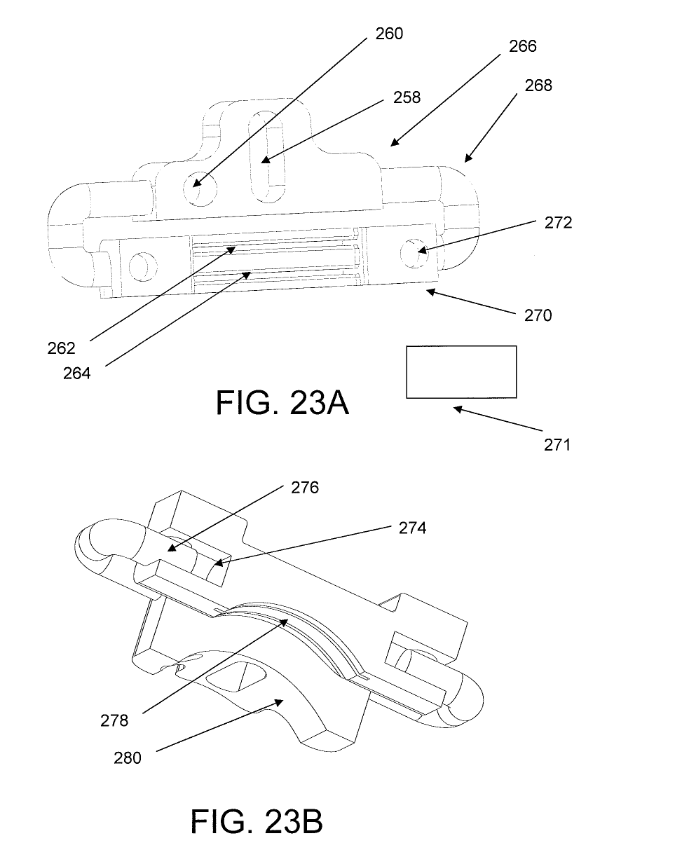

FIGS. 23A-B show perspective views of the talus bone cutter guide assembly 266. The talus bone cutter guide assembly 266 comprises two main bodies: the talus bone cutter guide top 268 and talus bone cutter guide bottom 270. Anatomically the talus joint surface can be curved. The talus bone cutter guide may be used to guide a thin rectangular type shaped saw type blade 271 to cut the talus joint surface substantially flat to conform to the surface 116 of the talar implant 102 (see FIG. 4A). This cutting of the talar joint surface could also be done to make room for the talar implant 102 and to create a bleeding bone surface for the talar implant 102 to fixate to the talar implant to the talus bone, as previously described herein. The talus bone cutter guide can be adjustable to conform to variable joint anatomies so that a repeatable substantially flat cut can be made by the surgeon to account for differences in anatomy from patient to patient.

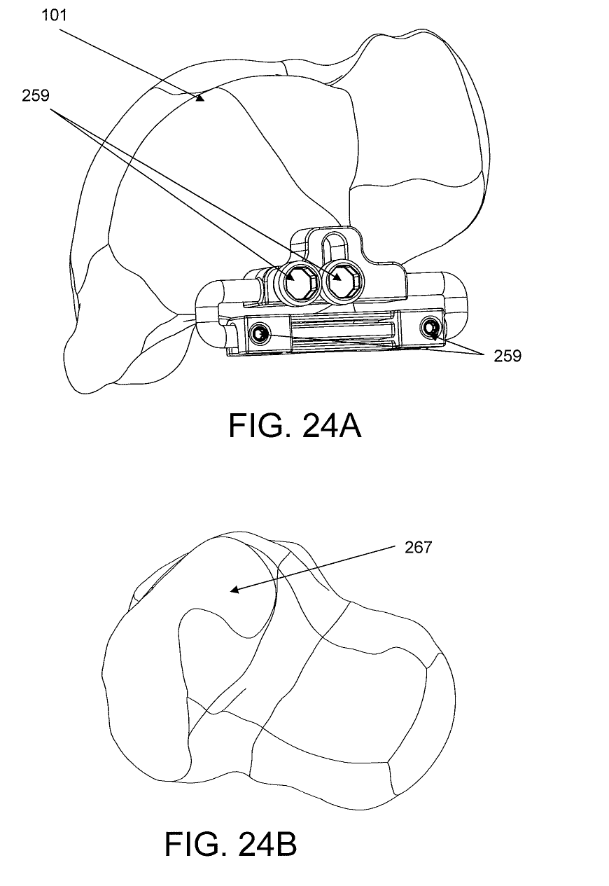

The talus bone cutter guide 266 may be first attached to the anterolateral undersurface of the talus after removing the anterior osteophytes/lip approximately 45° from the sagittal plane due to surgeon access constraints as shown in FIG. 24A. However, the attachment location of the talus bone cutter guide 266 to the talus bone 101 may be varied based on need.

First a set fastener 259, such as screw, nail, bolt, etc. may be inserted through the slotted aperture 258 of the talus bone cutter guide top 266 as shown in FIG. 24A. The screw aperture can be slotted so that the surgeon can position the talus bone cutter guide 226 in the transverse plane such that approximately 5 mm of bone can be removed from the talus. A guide type blade, similar to that of the cutter type blade, can be inserted through the talus bone cutter guide gauge slot 264. The guide type blade may be inserted down the length of the talus joint surface. If the guide type blade can be inserted down the entire length of the talus joint surface, the position indicates that the talus bone cutter guide can be set at the proper height relative to the transverse plane to remove about 5 mm of bone or perhaps slightly more than that amount of bone. This position may also set the depth of cut for the cutter type blade. If the talus bone cutter guide traverses down 10 mm to reach the end of the talus joint surface, the cutter blade can be set to cut to a depth of about 10 mm or perhaps slightly more than that amount of bone. The depth of cut can vary depending on variable joint anatomies from patient to patient.

Next a fastener, such as screw, nail, bolt, etc. may be inserted into aperture 260 to help the talus bone cutter guide 266 to be properly fixed in place as shown in FIG. 24A. This fixation of the talus bone cutter guide 266 to the talus bone through insertion of the fastener through the aperture 260 may not be necessary as adequate fixation of the talus bone cutter guide 266 to the talus bone may be provided by just a fastener installed through the slotted aperture 258 once adequately tightened in place.

FIG. 24B is a perspective view of the talus bone cut with the talus bone cutter guide 266 showing the cut surface 267.

The talus bone cutter guide bottom 270 can rotate about the axis of a post 276 of the talus bone cutter guide top 268 provided by the post 276 of the talus bone cutter guide top 268 and slot 274 of the talus bone cutter guide bottom 270. The rotational angle may be set similarly to that of how the distance of the subtalar joint prosthesis relative to the transverse plane can be set with the use of the talus bone cutter guide gauge slot 264 of the talus bone cutter guide bottom 270 and guide type blade.

The talus bone cutter guide top 268 can also slide or translate left and right in relation to the talus bone cutter guide bottom 270 also provided by the post and slot features previously described herein. The amount of translation can be adjusted to be bigger or smaller based on need. Once the surgeon is satisfied with the location of the talus bone cutter guide bottom 270 relative to the talus joint surface, the rotation and translation can be fixed in place by inserting and tightening fasteners into the fastener apertures 272. Interfacing surface pressure between fastener and subtalar joint prosthesis can provide proper fixation.

The cutter blade may then be inserted through the talus bone cutter guide slot 262 of the talus bone cutter guide bottom 270 and the bone can be cut substantially flat to a specified depth as shown in FIG. 19. A stop could be installed on the cutter blade set at the distance previously determined by the guide type blade to bottom out on the surface of the talus bone cutter guide bottom 270, setting the depth of cut. The depth of cut could also be restricted by the cutter type blade length itself or other technique and stay within the scope of the disclosure.

The slots 262 and 264 of the talus bone cutter guide bottom 270 can be substantially parallel to one another and approximately 5 mm in spacing. The space between the slots 262, 264 can be varied based on need to produce either a shallower or deeper cut of the talus bone. If the talar implant 102 was made thicker, then a deeper cut might be needed as well, and if the talar implant was made thinner, then a shallower cut could be needed. The depth of cut into the talus bone may also be dependent on variable joint anatomies from that may differ from patient to patient.

Surface 278 of the talus bone cutter guide bottom 270 and surface 280 of the talus bone cutter guide top 268 can be curved to be similar to that of the curvature of the talus bone where it can be attached. This may not be necessary, but might help to aid the attachment of the subtalar joint prosthesis to the bone surface. Furthermore, due to the plethora of possible ways of constructing a talus bone cutter guide, many of the components and features which comprise the talus bone cutter guide 266, as described herein, could be modified in size, shape, location, and number depending on need and stay within the scope of the disclosure.

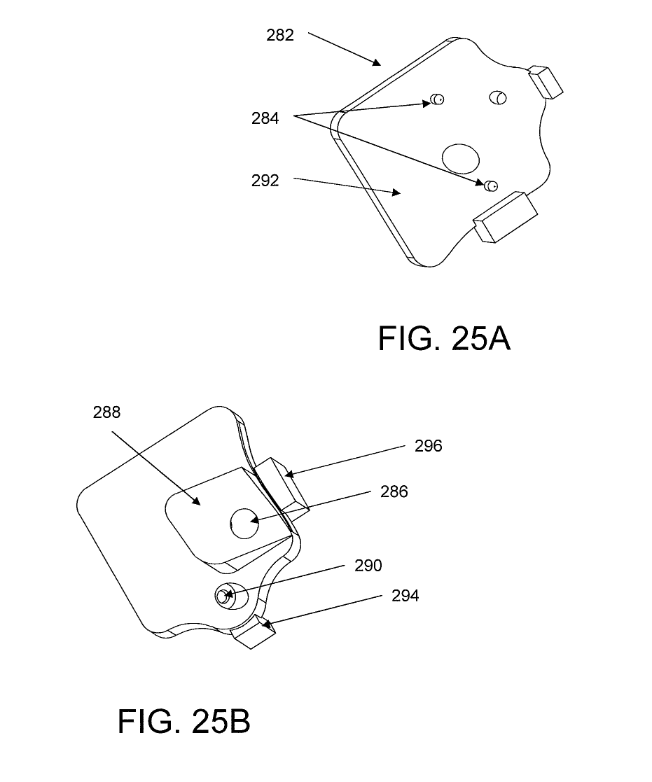

FIGS. 25A-B show exemplary views of a top dummy plate 282. The top dummy plate 282 can be used to guide the drill bit or similar cutting tool as it cuts an aperture in the talus bone joint surface at a specified angle and depth for the post 110 of the talar implant 102 (see FIG. 4A). The angle of an aperture 286 may be the same angle as that of the post 110 of the talar implant 102 that can be approximately 45° from both the sagittal and transverse planes. Other angles could be used. The diameter of aperture 286 might be slightly larger than that of the post 110 to help establish proper fit of the post 110 and provide a minimal amount of movement of the talar implant 102 relative to the talus bone and the calcaneus bone during installation, if necessary, to achieve proper spatial location of the subtalar joint prosthesis prior to fixation of the subtalar joint prosthesis.

The perimeter of the dummy plate 282 can be approximately the same size and shape as the talar implant 102. The fastener aperture 290 of the dummy plate 282 can be also in approximately the same location and approximately the same angle as that of the talus fastener aperture 112 of the talar implant 102 that can be approximately 45° from both the sagittal and transverse planes. However, the fastener aperture 290 can be set to have a slightly smaller diameter than that aperture 112 of the talar implant 102. This may not be necessary but the slightly smaller diameter may help when the talar implant 102 can be fastened in place allowing uncut bone material to be available to provide better short term fixation of the subtalar joint prosthesis. The location of the fastener aperture 290 could be moved as needed and stay within the scope of the disclosure.

There may also be two staking pins 284 of the dummy plate 282 of approximately similar size and approximately similar location to that of the staking pins 108 of the talar implant 102 (see FIG. 4A). The size, shape, and location of the staking pins 284 may be varied based on need. The staking pins 284 can be substantially orthogonal to a surface 292 of the dummy plate 282. The staking pins 284 may be angled approximately 45° from both the sagittal and transverse planes as those of the talar implant 102 can be. The staking pins 284 may not be angled approximately 45° from both the sagittal and transverse planes as those of the talar implant 102 can be. The staking pins 108 of the talar implant 102 can be the approximately same angle as the subtalar joint prosthesis may be installed at the approximately same angle as the post 110. However, the staking pins 284 of the top dummy plate 282 might be substantially orthogonal to surface 292 to facilitate the top dummy plate to be staked and aligned to the talus joint surface.

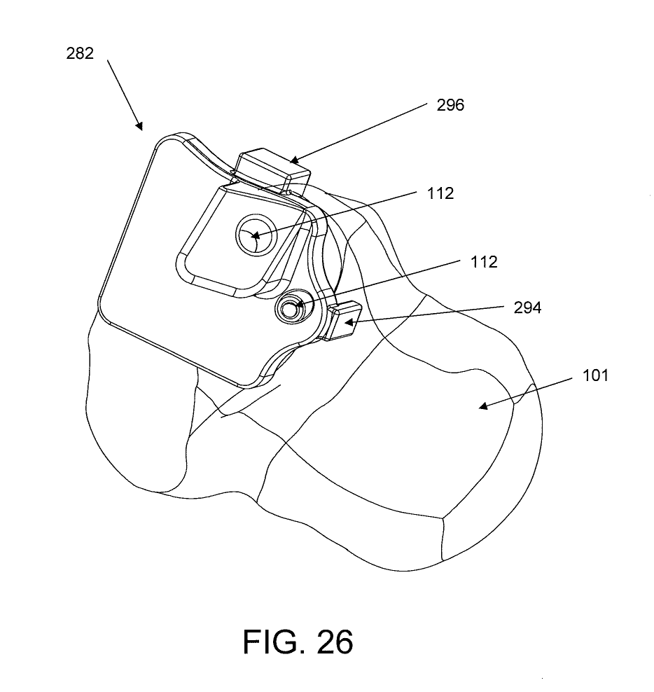

After the talus bone is cut substantially flat using the talus bone cutter guide 266, as previously described herein, the surgeon can align the dummy plate 282 to the talus using the alignment post 294 and the alignment post 296. The alignment post 294, 296 can be aligned with edges of the talus bone as shown in FIG. 26. The size, location, shape, and number of these the alignment posts could be varied based on need.

Next, the dummy plate 282 may be tapped in place by the surgeon driving the staking pins 284 into the talus bone to provide temporary fixation of the dummy plate 282 to the talus bone allowing a fastener to be installed through the aperture 290. Installing a fastener may not be necessary, but may help maintain temporary fixation such that the dummy plate 282 might not move when the aperture is cut for the post 110 of the talar implant 102.

Next, a drill type bit or cutting tool can be inserted through the aperture 286 of the dummy plate 282. The drill or cutting tool can have a stop feature which could rest on surface 288 of the dummy plate 282 setting the proper depth of the cut. The surface 288 can be angled such that it can be substantially orthogonal to the axis of aperture 286. The dummy plate 282 then may be removed and the talar implant can be checked for proper fit in the location prepared for installation of the talar implant. The talar implant 102 could be installed/attached to the talus at this time, but may not be necessary and installation of the talar implant may be performed later.

Due to the plethora of possible ways of constructing a dummy plate, many of the components and features which comprise the dummy plate 282, as described herein, could be modified in size, shape, location, and number depending on need and stay within the scope of the disclosure.

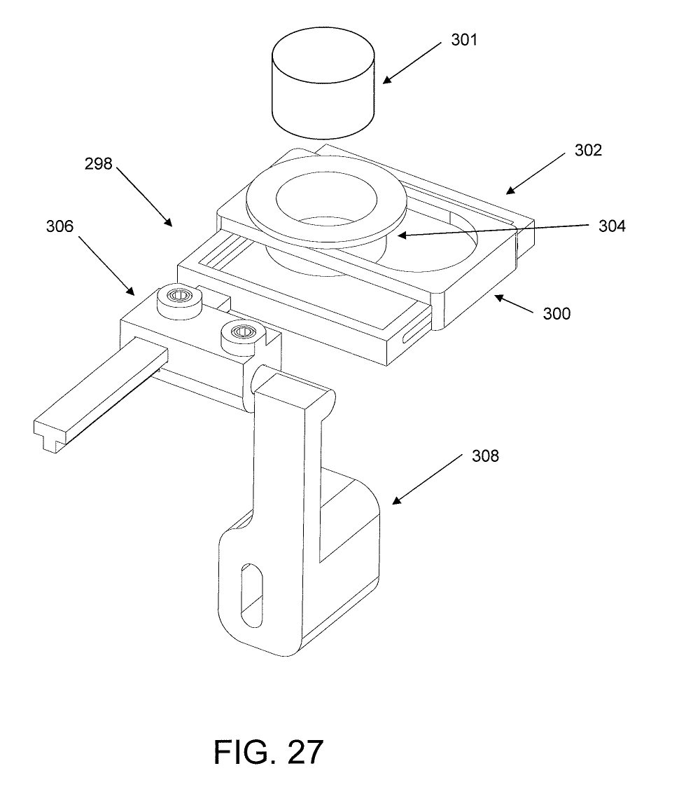

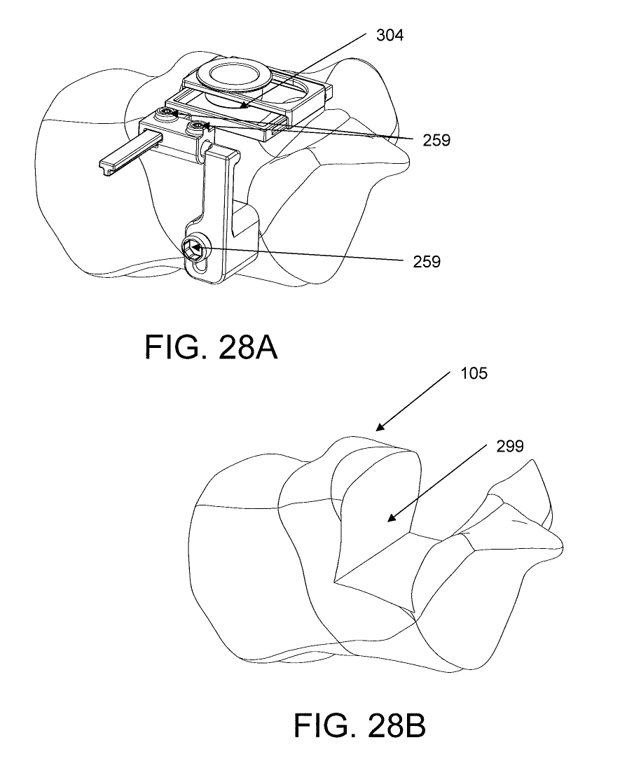

FIG. 27 shows a perspective view of the first calcaneus cutter guide 298. FIGS. 28A-B show the first calcaneus cutter guide 298 positioned for operation adjacent the calcaneus 105 and after cutting to expose the cut surface 299. The first calcaneus cutter guide 298 can be used to make the first bone cut of the base of the anterior surface of the calcaneus, just distal to the posterior facet of the calcaneus to a specified depth and the first calcaneus cutter guide 298 can be attached to the lateral side of the calcaneus at approximately the proximal base of the anterior surface or similar location. The first calcaneus cutter guide 298 may comprise 5 main bodies: coronal plane guide rail 300, sagittal plane guide rail 302, cutter guide 304, cutter guide top 306, and cutter guide bottom 308. Anatomically the calcaneus joint surface can be curved. The first calcaneus cutter guide 298 can be used to guide a mill type bit 301 angled at approximately 90° as it cuts the calcaneus joint surface to conform to surfaces of the calcaneal implant 106 as shown in FIG. 6B.

The calcaneal implant 106 surface 128 can sit flush on the surface made by the first calcaneus cutter guide 298. This cut and preparation can be also done to make room for the calcaneal implant and to create a bleeding bone surface for the calcaneal implant to fixate to as previously described herein. The first calcaneus cutter guide 298 might be adjustable to conform to variable joint anatomies to help aid a repeatable cut to be made by the surgeon to account for differences in anatomy from patient to patient.

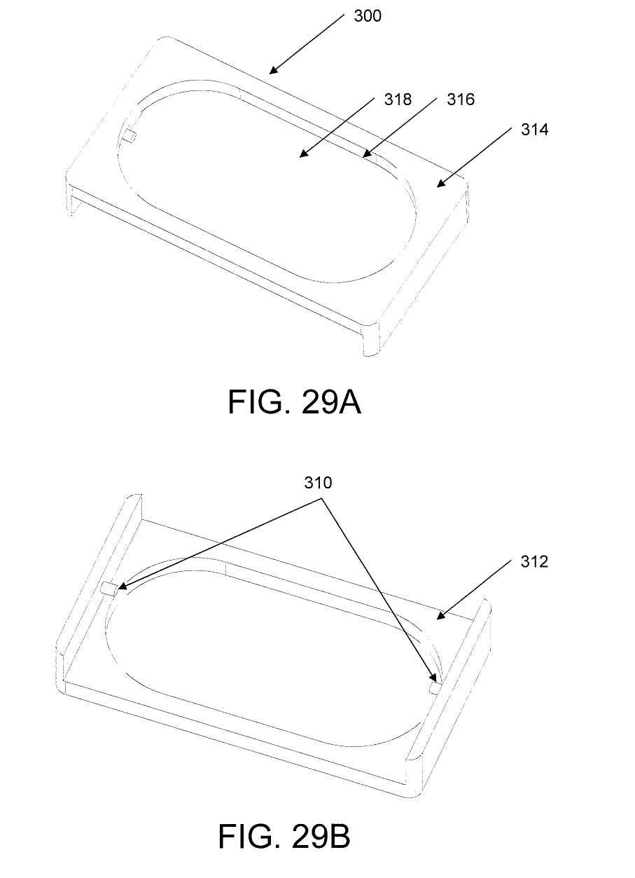

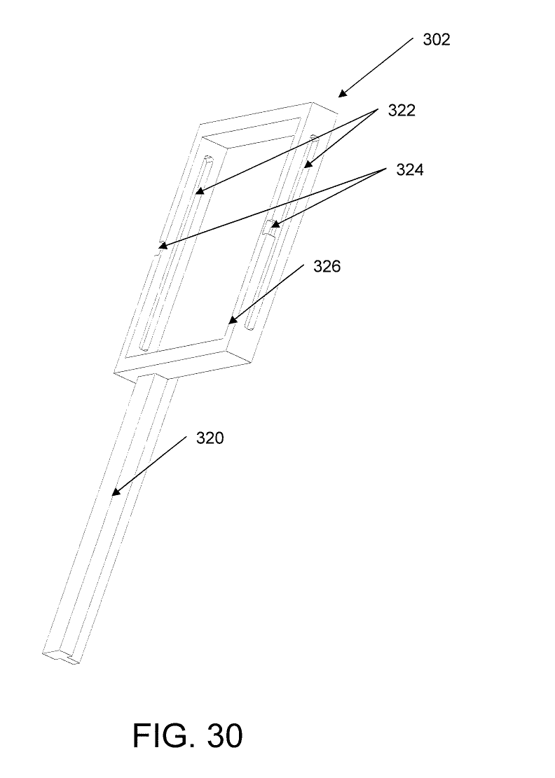

FIGS. 29A-B and FIG. 30 show perspective views of the coronal plane guide rail 300 and sagittal plane guide rail 302, respectively. The coronal plane guide rail 300 can be used to guide the mill type bit substantially parallel to that of the coronal plane. The width of the slot 318 may be dimensioned at a length twice that of the diameter of the mill type bit that may be inserted into the first calcaneus cutter guide 304. This sizing scheme may mean that only two passes may be needed to make the full bone cut resulting in a fast and repeatable cut. Of course, other sizing schemes are possible within the scope of the disclosure.

The diameter of the first calcaneus cutter guide 304 and width of the slot 318 of the coronal plane guide rail 300 could be made smaller or larger based on need. The first calcaneus cutter guide 304 could be made as a separate body, as shown, or could be attached directly to the mill type bit based on need and still be within the scope of the disclosure.

If the first calcaneus cutter guide 304 was attached directly to the mill type bit, it could be made adjustable to set the depth of cut. Contrariwise, if the first calcaneus cutter guide 304 was not attached to the mill type bit, a secondary stop feature might need to be added to the mill type bit to set the depth of cut. A set fastener could be added which might make the stop feature easily adjustable. Interfacing surface pressure between fastener and device could enable proper fixation. Either configuration might suffice and stay within the scope of the disclosure; however, attachment of the first calcaneus cutter guide 304 directly to the mill type bit could eliminate the need for an additional component.

The amount of relative slide or translation of the coronal plane guide rail 300 can be set by the length of the inner rectangle formed by the sagittal plane guide rail 302. The pins 310 of the guide rail 300 may be allowed to slide down the slots 322 of the guide rail 302. This arrangement may help the two guide rails remain attached to one another and also provide stability during the cutting operation. The number, shape, and size of the pins could be varied based on need.

The guide rail 300 can be assembled to the guide rail 302 by snapping the pins 310 over the top of the cut outs 324 of the guide rail 302 and into the slots 322. Once the two guide rails may be assembled, surface 312 of the guide rail 300 may rest on surface 326 of the guide rail 302. Surface 314 may provide a plane that the first calcaneus cutter guide 304 rests on during operation. Surface 316 may provide a plane that the first calcaneus cutter guide 304 travels along in the coronal plane during operation.

A beam 320 of the guide rail 302 can be inserted into the slot 334 of the first calcaneus cutter guide top 306. The beam 320 and the slot 334 might be “T” shaped to improve device stiffness, but the “T” shape may not be necessary and any shape depending on need. Even though the first calcaneus cutter guide 298 can be fastened to the side of the calcaneus, both guide rails can be adjusted in the coronal plane. This attachment of the first calcaneus bone cutter 292 to the side of the calcaneus with adjustment of guide rails in the coronal plane may be provided by the “T” shaped beam 320 of the guide rail 302 and “T” shaped slot 324 of the first calcaneus cutter guide top 306. The surgeon can move the guide rails until satisfied with the depth of cut relative to the coronal plane. Once satisfied with the location of the guide rails, a set fastener could be inserted into the aperture 328 of the first calcaneus cutter guide top 306. Interfacing surface pressure between fastener and the first calcaneus cutter guide could provide proper fixation. The length of travel could be varied based on need.

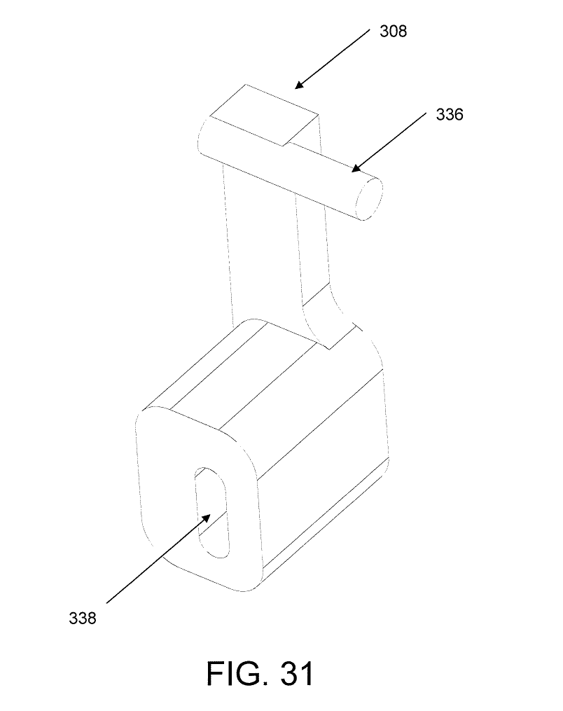

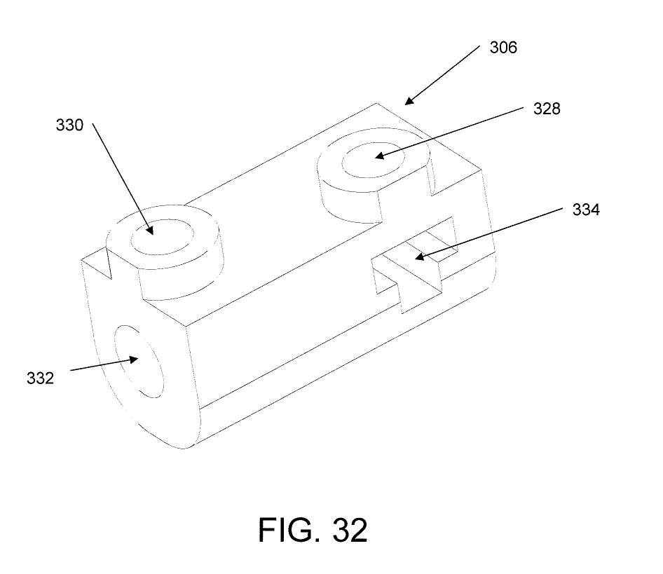

FIGS. 31-32 perspective views of the first calcaneus cutter guide bottom 308 and the first calcaneus cutter guide top 306, respectively. The aperture 328 and the slot 334 have already been described herein. Aperture 332 could allow the pin 336 of the first calcaneus cutter guide bottom 308 to be inserted within. Once the pin 336 is inserted within the aperture 332, the first calcaneus cutter guide top 306, guide rails 302 and 300, and the first calcaneus cutter guide 304 can both rotate about the axis formed by the aperture 332 of the first calcaneus cutter guide top, and the first calcaneus cutter guide 304 can translate in the coronal plane relative to the first calcaneus cutter guide bottom 308 as the first calcaneus cutter guide 298 can be fixed to the side of the calcaneus bone.

Once satisfied with the location of the guide rails 300 and 302, a set fastener could be installed into the aperture 330 of the first calcaneus cutter guide top 306 to fix translation and rotation as previously described herein. Interfacing surface pressure between fastener and the first calcaneus cutter guide 298 could provide proper fixation. The amount of translation and rotation of the first calcaneus cutter guide 298 could be varied based on need.

A slot 338 is provided by the first calcaneus cutter guide bottom 308. A fastener could be inserted in the slot 338 to attach the first calcaneus cutter guide 298 to the side of the calcaneus. The slot 338 provides additional slide or translation of the first calcaneus cutter guide 298 in the transverse plane. This translation or slide may help establish proper device spatial alignment of the first calcaneus cutter guide 298 relative to the calcaneus joint surface as calcaneus joint anatomies may vary from patient to patient.

Due to the plethora of possible ways of constructing a first calcaneus cutter guide, many of the components and features which comprise the first calcaneus cutter guide 298, as described herein, could be modified in size, shape, location, and number depending on need and stay within the scope of the disclosure.

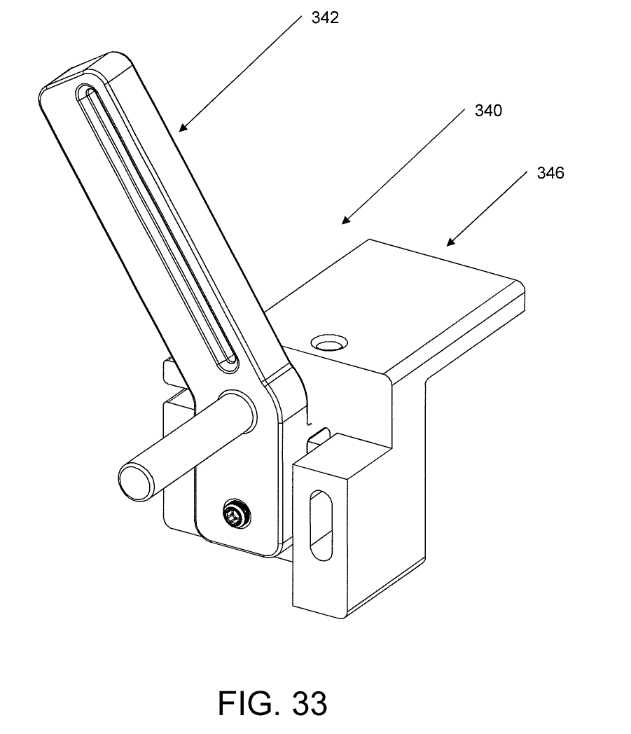

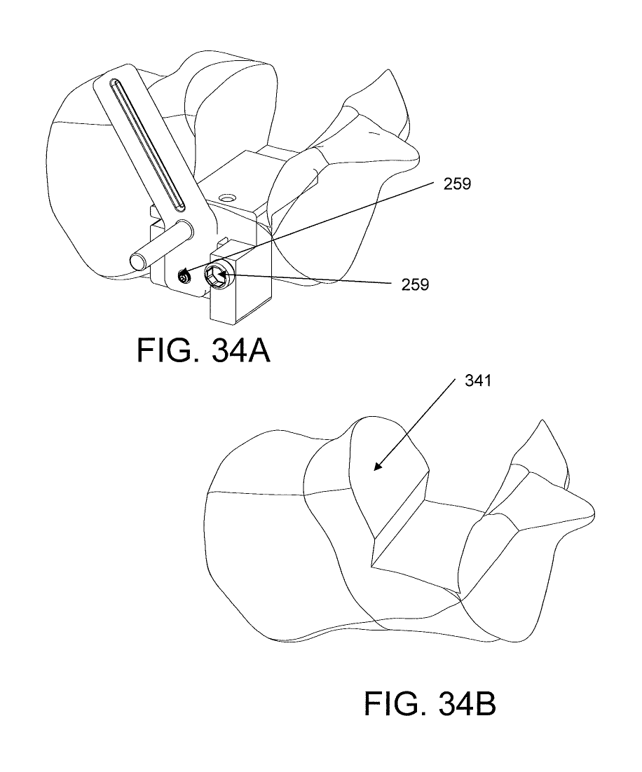

FIG. 33 shows a perspective view of the second calcaneus cutter guide 340. FIGS. 34-37 provide further details regarding the second calcaneus cutter guide 340. The first calcaneus cutter 340 guide may comprise three main bodies: a second calcaneus angle cutter guide 342 for the second cut and a base plate 346 to which the second cutter guide 342 attaches. The bone cutter guide 340 can be used to make the second bone cuts of the calcaneus. The second cut can be made to the posterior facet surface of the calcaneus with the second cutter guide 342 installed. The second calcaneus angle cutter guide 342 can be attached to the side of the calcaneus in a substantially similar location to that of the first calcaneus cutter guide 298 that was previously installed and then removed, as shown in FIGS. 28A-B. A cut bone surface 299 from the cut performed with the first calcaneus cutter guide 298 may be used. A cut bone surface 341 from the cut performed by the 2nd calcaneus cutter guide 340 is shown in FIG. 34B. The location of attachment of the second calcaneus cutter guide 340 may be varied depending on need and does not necessarily need to be in substantially the same location as that of the first calcaneus cutter guide 298.

Anatomically the calcaneus joint surface can be curved. The second calcaneus cutter guide 340 can be used to guide a substantially flat saw type blade as it cuts the calcaneus joint surface to conform to surfaces of the calcaneal implant 106 that are contactingly adjacent the calcaneus when the calcaneal implant 106 is installed. This cut to the calcaneus can be also done to make room for the calcaneal implant and to create a bleeding bone surface for the calcaneal implant to fixate to as previously described herein. The second calcaneus cutter guide 340 can be adjustable to conform to variable joint anatomies to help enable a repeatable cut that can be made by the surgeon to account for differences in anatomy from patient to patient.

The angle of a guide slot 348 in the second calcaneus cutter guide 340 can be set to be substantially the same angle as the angle of the surface of the calcaneal implant 106 (see FIG. 6A) that is contactingly adjacent the calcaneus when the calcaneal implant 106 is installed. The angle of the second calcaneus cut can be set to approximately 57° from the coronal plane or approximately 33° from the transverse plane or both approximately 57° from the coronal plane and approximately 33° from the transverse plane. The magnitude of the angle for the cut produced through the operation of the second calcaneus cutter guide 340 can be varied based on need. However, the angle might be set to both 57° from the coronal plane and 33° from the transverse plane to better conform to the existing calcaneus joint surface anatomy which may help reduce the overall amount of bone removal that could be needed during installation of the calcaneal implant 106. This angulation of the second calcaneus cut also may be done to allow the joint axis of the subtalar joint prosthesis 100 to be at approximately 45°, as shown in FIG. 1, which may better conform to the native joint axis of the calcaneus bone as previously described herein.

The angle of the first cut bone surface to the second cut bone surface can be fixed at approximately 57° from the coronal plane. The angle can be provided by surface 372 of the base plate 346 as the second calcaneus cutter guide 340 rests on the first calcaneus cut surface. This orientation may help such that when the second calcaneus surface can be cut, the first and second calcaneal cut surfaces may be approximately 57° from one another. The depth of cut of the second calcaneus cut can be set based on how deep the first calcaneus cut can be made. The deeper the first calcaneus cut, the deeper typically the second calcaneus cut can be made depending on individual joint anatomies, etc. The depth of the calcaneus cuts, in turn, can control the amount of bone that may be removed for installation of the calcaneal implant 106.

The second calcaneus angle cutter guide 342 can be made to be removable from the base plate 346. This reversible removability may be done to make room for the surgeon when making the cuts in the calcaneus.

The cutting procedure can begin with first attaching the base plate to the side of the calcaneus. The base plate 346 can be located to the cut surface of the calcaneus provided by surfaces 372 and 376, and the base plate 346 may be tapped down in place. The stake 374 may provide temporary fixation to the calcaneus bone while a fastener, such as a screw or nail, can be installed through the slotted aperture 368 of the base plate 346 as shown into the calcaneus bone. The second calcaneus angle cutter guide 342 could be installed either before or after the base plate 346 can be attached to the calcaneus bone. However, it might be easier to install the base plate 346 before the second calcaneus angle cutter guide 342 can be attached to the base plate.

The first calcaneus cutter guide 298 can be installed by first sliding the arm 356 through the slot of the base plate 346 formed by surfaces 366 and 378. To aid installation, the surgeon could use the handle 352 of the second calcaneus angle cutter guide 342, although it may not be needed. Surface 358 of the second calcaneus angle cutter guide 342, could rest on surface 366 of the base plate 346 once installed.

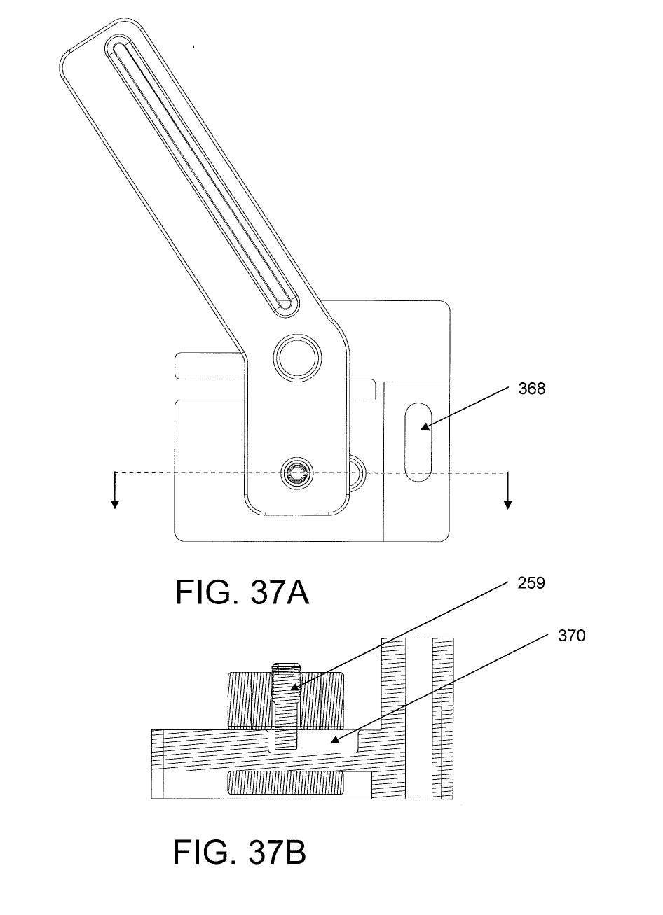

Next, a threaded set fastener can be installed through aperture 354 of the second calcaneus angle cutter guide 342 and slot 370 of the base plate 346. Section 37A-37A of FIG. 37B shows the set fastener can be installed and may fix the second calcaneus angle cutter guide 342 to the base plate 346. Interfacing surface pressure between fastener and the second calcaneus bone angle cutter guide 342 could provide proper fixation.

After the second calcaneus angle cutter guide 342 may be installed to the base plate 346, a small amount of relative slide or translation in the coronal plane can be provided between the two components by the set fastener and slot 370 of the base plate 346 also shown in section 37A-37A of FIG. 37B. The set fastener may be bottomed out on the surface of the slot furthest from the slotted aperture 368 of the base plate 346. This position may help the surgeon to remove the right amount of bone during the second cut by helping establish proper conformation of the calcaneal implant surface 136 to that of the cut bone surfaces.

The calcaneus then may be cut to a specified depth relative to the sagittal plane. The depth of cut could be controlled by an adjustable stop added to the substantially flat saw type blade. The second calcaneus angle cutter guide 342 and base plate 346 can be fixed to the side of the calcaneus bone as well as the cut bone surface for reference.

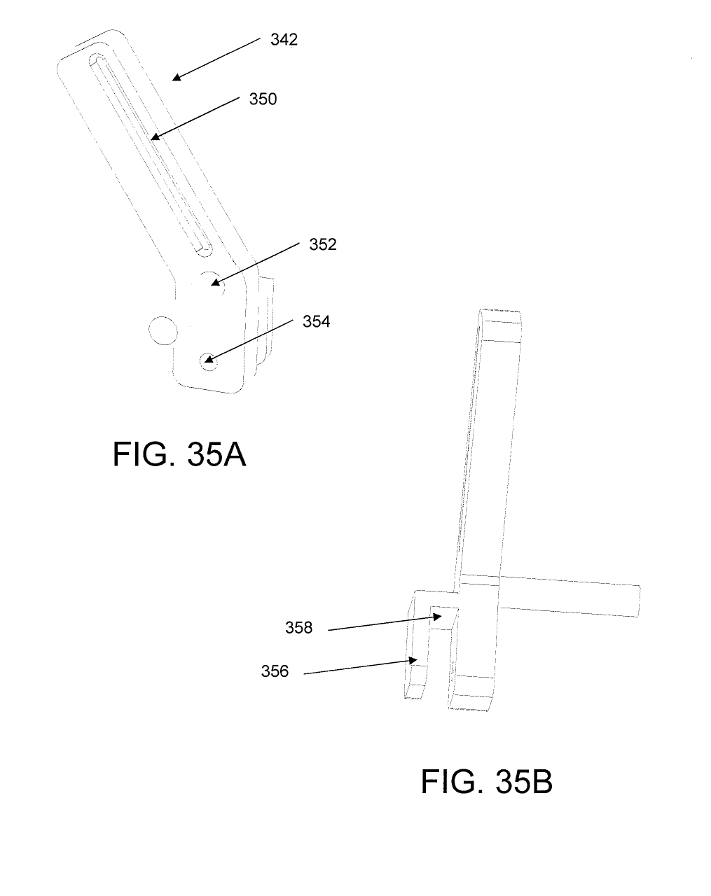

FIGS. 35A-B show perspective views of the second calcaneus angle cutter guide 342. Many of the features of the second calcaneus angle cutter guide 342 have already been described herein. The chamfer 350 can be used to allow ease of insertion of the thin shaped saw like blade, although may not be necessary. The thickness of the saw blade could be similar to that of the width of the guide slot 348, such that the guide slot might properly guide the saw type blade during cutting. However, the width of this slot could be varied based on need.

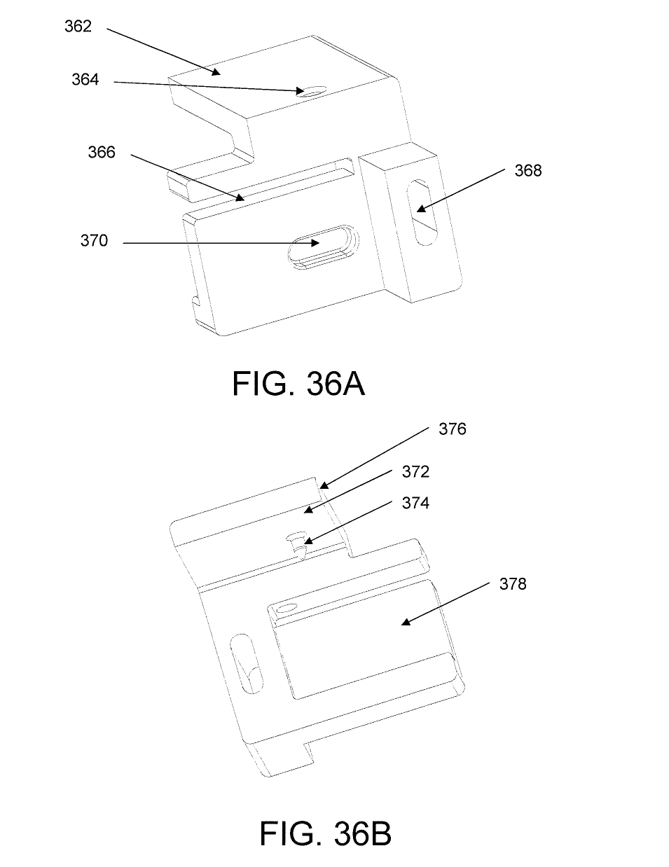

FIGS. 36A-B show perspective views of the base plate 346. Many of the features of the base plate 346 have already been described herein. The aperture 368 may be slotted to allow for the fastener to have some relative slop or translation in the transverse plane. This translation in the transverse plane might help to aid in installation to accommodate for variable joint anatomies and depths of first calcaneus cuts, although it may not be necessary. The overall size and shape of the base plate 346 could be modified based on need.

FIG. 38 shows a perspective view of the calcaneal implant 106 installed to the calcaneus bone, after all cuts is made for reference. A fastener can be installed through aperture 124 of the calcaneal implant 106 (see FIG. 6A). The fastener might provide temporary fixation of the subtalar joint prosthesis so that bony ingrowth can occur to provide long term fixation of the subtalar joint prosthesis as previously described herein.

FIG. 39 shows a perspective view of the talar implant 102 installed to the talus bone, after the cut is made for reference. A fastener can be installed through aperture 112 of the talar implant 102 (see FIG. 4B). The fastener might provide temporary subtalar joint prosthesis fixation so that bony ingrowth can occur to provide long term fixation of the subtalar joint prosthesis as previously described herein. Either the talar or calcaneal implant could be installed first as desired based on need.

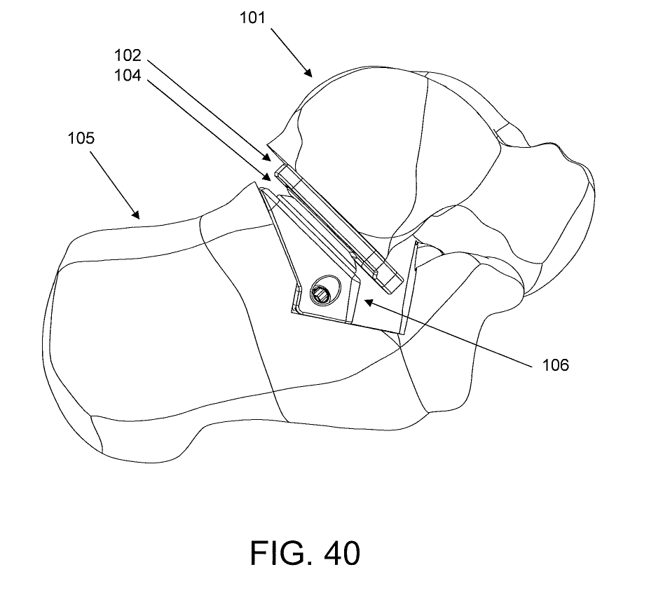

FIG. 40 shows a side view of the sheet 104 installed between the talar implant 102 and calcaneal implant 106 forming the subtalar joint prosthesis 100. The talus and calcaneus bones can be retracted with the distracter 144 in order to install the sheet 104. The surgeon can then check the subtalar joint gap of the patient by removing the distraction between the talus and calcaneus bones. The joint gap can be easily adjusted to be larger or smaller by installing sheets 104 that can be thicker or thinner, respectively. The talus and calcaneus bones could be distracted again and variable thickness sheets could be installed until the proper or desired joint gap is achieved. Once the proper or desired joint gap is achieved, the distracter then can be removed and the incision can be closed using standard surgical practices.

FIG. 41 is a side view looking from the lateral aspect showing the subtalar joint distracted by the distracter 144. The distracter main body 148 is attached to the talus bone 101 posterior to where the fibula would be located. The fibula is left intact during installation of a subtalar prosthesis by a lateral approach using the set of tools for installation disclosed. Only the talus bone and the calcaneus need be cut during installation of the subtalar process using the set of tools for installation disclosed. The distracter front body 146 is attached to the calcaneus 105. The talus bone 101 and the calcaneus 105 are distracted, which means separated without rupture of their binding ligaments, using the distracter 144 to facilitate implanting the subtalar prosthesis.

While the distracter 144 will typically be used for installation of the subtalar prosthesis, the distracter 144 may have other uses. For example, the distracter 144 may be used to distract bones in the setting of surgery for fusion, nonunion repair, etc.

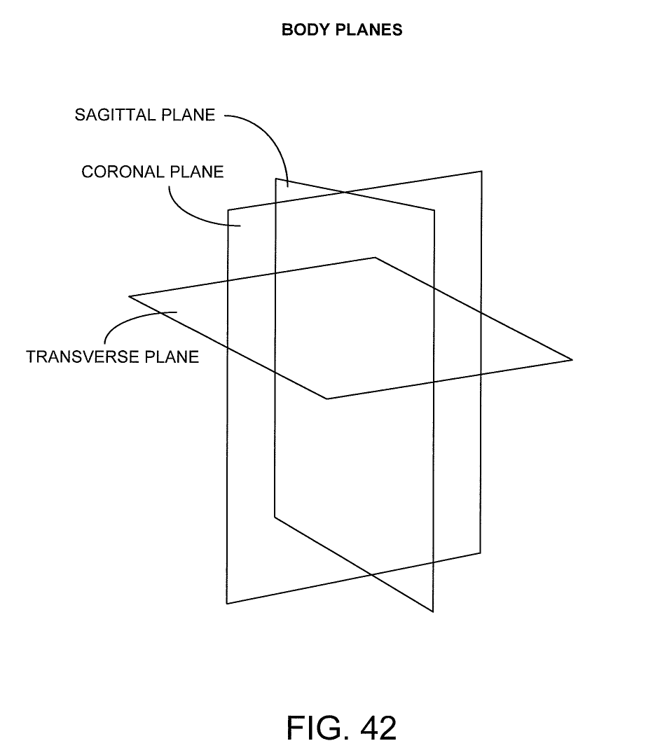

FIG. 42 demonstrates the anatomic planes as understood by one skilled in the art. Furthermore, the terms anterior, posterior, medial, lateral, and other anatomic terms, as used herein, have the standard meaning as understood by one skilled in the art. Anterior means towards the front. Posterior means towards the back. Medial means towards the midline. Lateral means towards the side. A combination of terms may be used and understood, such as posteromedial and anterolateral.

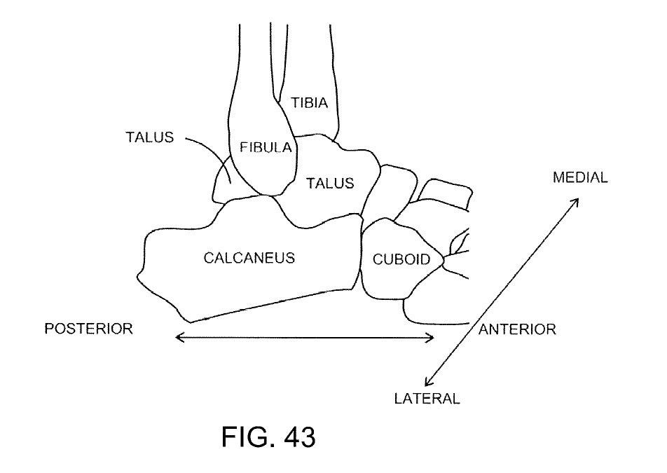

FIG. 43 shows a lateral view of the talus and calcaneus anatomy. In a standard anatomic position, the tibia is medial to the fibula.

All of the assemblies and components may be made out of various different types of materials such as stainless steel or titanium alloy based on need, cost, etc. Polymers and other suitable materials may be used, also. All of the assemblies and components may be manufactured using various processes such as casting, machining, etc. based on need, cost, etc. Various component surfaces may also be plated or various components heat treated as needed to reduce wear, improve desired material properties, etc. Various components may be made biocompatible.

The subtalar joint prosthesis may generally comprise three main bodies: a talar implant, a calcaneal implant, and a sheet positioned between the talar and calcaneal implants. The talar implant can be fastened to the talar undersurface, and the calcaneal implant can be fastened to the calcaneal top surface. The sheet may be allowed to float or translate relatively freely in relation to the talar and calcaneal implants. The sheet provides a curvilinear surface that allows rotation in the transverse, sagittal, and coronal planes between the talar and calcaneal implants thus enabling three dimensional joint motion between the talus and calcaneus bones mimicking the joint motion of the native subtalar joint of the human body. The curvilinear surface may be continuously curvilinear, as may other curvilinear surfaces disclosed.

This disclosure offers a unique geometry specifically designed for use as a joint replacement for the subtalar joint, as well reproducing the motion of the native subtalar joint. This disclosure also includes a surgical procedure for installing the implant in the body, as well as tools to assist in performing the surgical procedure with the disclosed subtalar prosthesis or other subtalar prosthesis.

It is to be understood that even though numerous characteristics and advantages of various embodiments of the present disclosure have been set forth in the foregoing description, together with details of the structure and function of various embodiments of the disclosure, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. An apparatus comprising:
a first implant configured for attachment to a talus bone;
a second implant configured for attachment to a calcaneus bone; and
a sheet configured to be disposed between the first implant attached to the talus bone and the second implant attached to the calcaneus bone, wherein the sheet has rotational and translational motion with respect to the first implant and the second implant in order to facilitate substantially normal kinematic motion in the subtalar joint.

2. The apparatus of claim 1, wherein the sheet provides a curvilinear surface that engages a curvilinear surface of the first implant to facilitate rotational motion of the first implant in relation to the second implant, and the sheet provides a substantially two-dimensional planar surface opposing the curvilinear surface of the sheet, wherein the substantially two-dimensional planar surface of the sheet engages a substantially two-dimensional planar surface of the second implant to facilitate translation of the first implant in relation to the second implant.

3. The apparatus of claim 2, wherein the sheet provides a post and the second implant provides an indentation, such that the interaction between the post and the indentation restricts translation of the sheet in relation to the second implant.

4. The apparatus of claim 2, wherein the curvilinear surface of the sheet has a convex protrusion that is configured to be complementary to a concave surface provided by the curvilinear surface of the first implant.

5. The apparatus of claim 4, wherein interaction between the convex protrusion and the concave surface is configured to restrict motion between the sheet and the first implant.

6. The apparatus of claim 1, wherein the sheet provides a curvilinear surface that engages a curvilinear surface of the second implant to facilitate rotational motion of the second implant in relation to the first implant, and the sheet provides a substantially two-dimensional planar surface opposing the curvilinear surface of the sheet, wherein the substantially two-dimensional planar surface of the sheet engages a substantially two-dimensional planar surface of the first implant to facilitate translation of the second implant in relation to the first implant.

7. The apparatus of claim 6, wherein the sheet provides a post and the first implant provides an indentation, such that the interaction between the post and the indentation restricts translation of the sheet in relation to the first implant.

8. The apparatus of claim 1, wherein the sheet is made from material comprising a biocompatible polymer.

9. The apparatus of claim 1, wherein the first implant and the second implant are each secured by a mechanical fastener in relative fixation to the talus bone and the calcaneus bone, respectively, to promote short-term fixation.

10. The apparatus of claim 1, wherein the first implant and the second implant are each prepared with a biocompatible surface that promotes bone growth from the talus bone and the calcaneus bone, respectively, to promote long-term fixation and stability.

11. An apparatus comprising:
a distracter main body configured for attachment to a talus bone; and
a distracter front body configured for attachment to a calcaneus, wherein the distracter main body attached to the talus bone and the distracter front body attached to the calcaneus cooperate to move between a first position and a second position to distract the talus bone from the calcaneus without contacting bones other than the talus bone and the calcaneus during attachment of the distracter main body to the talus bone and attachment of the distracter front body to the calcaneus.

12. The apparatus of claim 11, further comprising:
a wrench type mechanism coupled to the distracter main body and the distracter front body, the wrench type mechanism configured to move between the first position and the second position.

13. The apparatus of claim 11, wherein the distracter main body provides a first set of gear type teeth and the distracter front body provides a second set of gear type teeth, and engagement of the first set of gear type teeth by the second set of gear type teeth is configured to move between the first position and the second position.

14. The apparatus of claim 11, further comprising:
a button coupled to both the distracter main body and the distracter front body, wherein in a first activation position the button activates a mechanism to move between the first position and the second position, and in a second activation position the button activates a mechanism to move between the second position and the first position.

15. The apparatus of claim 14, wherein in the first activation position the button is configured to activate a wrench type mechanism to extend the distracter main body in relation to the distracter front body in a first direction, in the second activation position the button is configured to activate the wrench type mechanism to retract the distracter main body in relation to the distracter front body in a second direction which opposes the first direction, in a third activation position the button is configured to allow freedom of travel to extend or retract the distracter main body in relation to the distracter front body, and in a fourth activation position the button is configured to lock the wrench type mechanism to substantially eliminate extension or retraction of the distracter main body in relation to the distracter front body.

16. The apparatus of claim 11, further comprising:
a wrench type mechanism coupled to the distracter main body and the distracter front body, wherein the wrench type mechanism is configured to move between the first position and the second position; and
a bias element configured to activate the wrench type mechanism in a first activation position and deactivate the wrench type mechanism in a second activation position, wherein activation of the wrench type mechanism is configured to move between the first position, and deactivation of the wrench type mechanism is configured to eliminate movement between the first position and the second position.

17. The apparatus of claim 11, wherein the talus bone is distracted from the calcaneus approximately 2 inches with movement from the first position to the second position.

18. The apparatus of claim 11, wherein the second position is configured to facilitate attachment to the talus bone of a talus bone cutter guide configured to prepare the talus bone for installation of a talar implant, and wherein the second position is configured to facilitate attachment to the calcaneus of a calcaneus bone cutter guide configured to prepare the calcaneus for installation of a calcaneal implant.

19. A method comprising, steps of:
exposing a lateral dissection of a subtalar joint to expose a talus bone and calcaneus;
fastening a distracter to lateral sides of the talus bone and the calcaneus;
distracting the talus bone from the calcaneus by the distracter;
removing the anterior osteophytes from the talus bone;
fastening a talus bone cutter guide to the anterolateral undersurface of the talus;
inserting a first substantially flat shaped saw type blade into the talus bone cutter guide to cut the talus bone substantially flat to conform to a surface of a talar implant;
fastening a dummy plate, having the same geometry of the talar implant, to the cut talus bone;
preparing a post hole in the talus bone using a guide aperture in the dummy plate;
fastening a first calcaneus cutter guide to the lateral side of the calcaneus at a proximal base of the anterior surface;
milling down to a specified depth a base of the anterior surface of the calcaneus distal to the posterior facet of the calcaneus;
fastening a second calcaneus cutter guide to the lateral side of the calcaneus; and
cutting the calcaneus with a second substantially flat shaped saw type blade using the second calcaneus cutter guide to direct location of a cut in the calcaneus, wherein only the only bones damaged in the steps are the talus bone and the calcaneus.

20. The method of claim 19, further comprising:
inserting the talar implant, wherein the talar implant has a post that lines up with the post hole, and the talar implant is inserted contactingly adjacent the cut talus bone;
inserting a calcaneal implant, wherein the calcaneal implant is inserted contactingly adjacent the milled down calcaneus and the cut calcaneus;
inserting a sheet between the talar implant and the calcaneal implant;
allowing the sheet become contactingly adjacent both the talar implant and the calcaneal implant.
